(19) United States
(12) Patent Application Publication
(10) Pub. No.: US 2019/0117663 A1
(43) Pub. Date: Apr. 25, 2019

(54) COMPOSITIONS AND METHODS FOR TREATING CANCER

(71) Applicant: CASE WESTERN RESERVE UNIVERSITY, Clevland, OH (US)

(72) Inventors: Analisa Difeo, Cleveland, OH (US); Goutham Narla, Cleveland, OH (US); Amy D. Armstrong, Cleveland, OH (US)

(21) Appl. No.: 16/091,492

(22) PCT Filed: Apr. 4, 2017
(86) PCT No.: PCT/US17/25945

§ 371 Date: Oct. 4, 2018

Related U.S. Application Data
(60) Provisional application No. 62/318,066, filed on Apr. 4, 2016.

(51) Int. Cl. A61K 31/538; A61K 31/404; A61K 31/553; A61K 31/5513; A61K 31/55; A61P 35/00; A61K 31/5415; A61K 31/5383; A61K 31/519; A61K 31/407; A61K 31/437; A61K 31/18

(52) U.S. Cl. CPC A61K 31/538; A61K 31/404; A61K 31/553; A61K 31/5513; A61K 31/55; A61K 31/18; A61K 31/5415; A61K 31/5383; A61K 31/519; A61K 31/407; A61K 31/437; A61P 35/00

(57) ABSTRACT

A method of treating cancer in a subject in need thereof includes administering to the subject therapeutically effective amounts of a PP2A activator and a BER inhibitor, such a PARP inhibitor.

### RELATED APPLICATION

This application claims priority from U.S. Provisional Application Ser. No. 62/318,066, filed Apr. 4, 2016, the subject matter of which is incorporated herein by reference in its entirety.

### BACKGROUND

Protein kinases have gained acceptance as therapeutic targets and have become a major focus of drug development efforts in oncology, with hundreds of inhibitors either in the pipeline or already in the clinic. Protein phosphatases, on the other hand, have been largely ignored for drug development because of their reputed lack of substrate specificity and the toxicity associated with natural products discovered as potent active site inhibitors.

Protein phosphatase 2A (PP2A) is one of the four major serine threonine phosphatases and is implicated in the negative control of cell growth and division. PP2A holoenzymes are heterotrimeric proteins composed of a structural subunit A, a catalytic subunit C, and a regulatory subunit B, dephosphorylates key oncogenic signaling proteins to function as a tumor suppressor. The PP2A protein phosphatase is a ubiquitous and conserved phosphatase with broad substrate specificity and diverse cellular functions. In contrast to the narrow substrate specificity of protein kinases, PP2A interacts with multiple substrates, and therefore its activation is, in effect, a combination therapy that coordinately inhibits multiple signaling pathways, including oncogenic signaling pathways. Among the targets of PP2A are proteins of oncogenic signaling cascades, such as Raf, MEK, AKT, ERK and FOXO.

### SUMMARY

This application relates to compositions and methods for treating cancer and particularly relates to the use of PP2A activators in combination with base excision repair (BER) inhibitors, such as PARP inhibitors, and/or pharmaceutical compositions comprising the same, to treat cancer in subjects in need thereof.

In some embodiments, a method of treating cancer in a subject in need thereof can include administering to the subject therapeutically effective amounts of a PP2A activator and a PARP inhibitor. In some aspects, the subject can be a human subject. In other aspects, the cancer can be characterized by cancer cells in which PP2A expression is reduced and/or Plk1 is overexpressed.

In still other aspects, the cancer can be resistant to treatment with a PARP inhibitor. For example, the cancer can include those that are BRCA1/2 wild type, that is, the subject has a BRCA genotype not associated with an increased risk of hereditary breast-ovarian cancer syndrome.

In other embodiments, the subject or cancer can have a BRCA1/2 mutation, that is, the subject has a BRCA genotype associated with an increased risk of hereditary breast-ovarian cancer syndrome.

In still other embodiments, the cancer treated with the PP2A activator and BER inhibitor can be ovarian cancer or breast cancer.

In some embodiments, the PP2A activator can include a small molecule that promotes and/or induces PP2A activation. For example, the PP2A activator can be triycyclic neuroleptic compound or a derivative thereof.

In some embodiments, the BER inhibitor is a PARP inhibitor.

In another embodiment, a method for treating cancer in a subject in need thereof includes administering to the subject a therapeutically effective amount of a pharmaceutical composition comprising a coformulation of a PP2A activator, a PARP inhibitor and a pharmaceutically acceptable carrier thereof.

### BRIEF DESCRIPTION OF THE FIGURES

The foregoing and other features of the application will become apparent to those skilled in the art to which the application relates upon reading the following description with reference to the accompanying drawings, in which:

FIGS. 1(A-B) illustrate: (A) cluster analysis results of phosophoproteomics data generated from cells treated with SMAPs; and (B) an immunoassay showing SMAP-061 rapidly and potently induces cleaved PARP and yH2AX, and degrades Plk1 and its downstream target, cyclin B1.

FIGS. 2 (A-B) illustrate: (A) plots showing stable expression of activated Plk1 (T210D) blocks SMAP-061 effects on apoptosis as measured through annexin V staining; and an immunoassay showing cleaved PARP and γH2AX induction.

FIGS. 3(A-C) illustrate immunoassays and a graph showing Plk1 degradation is a direct effect of PP2A activation. (A) Okadaic acid a pharmacological inhibitor of PP2A, rescues SMAPs effects on Plk1 degradation and the induction of cleaved PARP and pH2AX. (B) Phospho-MPM2-FITC analysis of mitosis reveals that SMAP-061 blocks nocodazole induced G2/M phase arrest. (C) SMAP-061 abrogates nocodazole activation of pPLK1 and total Plk1 and combination of nocodazole and SMAP-061 leads to enhanced γH2Ax induction.

FIGS. 4(A-C) illustrate plots and an image of colony formation assays showng SMAPs synergize with PARP inhibitors. (A) Isobologram analysis and combination index calculations reveal that SMAP-061 treatment synergizes with the PARP inhibitor, Olaparib. (B) Colony formation assays confirm that the combinations of the two drugs are significantly more potent at reducing cell survival then the 2 drugs alone. (C) Western blot analysis reveal that the Olaparib plus SMAP-061 treatment results in a greater induction of γH2AX and cleaved PARP as well as a more pronounced decrease in Plk1 and Cyclin B1.

FIG. 5 illustrates a graph showing HGSOC PDX drugs studies with SMAP-061 and Olaparib. SMAP-061 significantly sensitized a BRCA1/2 wildtype PDX tumor to the PARP inhibitor, Olaparib. Graph depicts fold change in tumor volume at day 16 which was the end of the study.

FIGS. 6(A-C) illustrate graphs showing the results of drug studies with SMAP-061 and Olaparib on germline BRCA1 high grade serious ovarian cancer patients.

### DETAILED DESCRIPTION OF THE INVENTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the application.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “agent” and “drug” are used herein to mean chemical compounds, mixtures of chemical compounds, biological macromolecules, or extracts made from biological materials, such as bacteria, plants, fungi, or animal particularly mammalian) cells or tissues that are suspected of having therapeutic properties. The agent or drug may be purified, substantially purified, or partially purified.

The term “antineoplastic” is used herein to mean a chemotherapeutic intended to inhibit or prevent the maturation and proliferation of neoplasms (tumors) that may become malignant, by targeting the DNA.

The terms “comprising”, “including”, and variations thereof do not have a limiting meaning where these terms appear in the description and claims.

The terms “treatment,” “treating,” and “treat” refer to any treatment of cancer, (e.g., breast, ovarian, leukemia, prostate cancer, and non-small-cell lung cancer) in a subject including, but not limited to, inhibiting disease development, arresting development of clinical symptoms associated with the disease, and/or relieving the symptoms associated with the disease. However, the terms “treatment,” “treating,” and “treat” are not necessarily meant to indicate a reversal or cessation of the disease process underlying the cancer afflicting the subject being treated. Such terms indicate that the deleterious signs and/or symptoms associated with the condition being treated are lessened or reduced, or the rate of progression or metastasis is reduced, compared to that which would occur in the absence of treatment. A change in a disease sign or symptom can be assessed at the level of the subject (e.g., the function or condition of the subject is assessed), or at a tissue or cellular level. In accordance with the present invention, desired mechanisms of treatment at the cellular level include, but are not limited to one or more of a reduction of cancer cell process extension and cell migration, apoptosis, cell cycle arrest, cellular differentiation, or DNA synthesis arrest.

The term “prevention” includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those having an enhanced risk of developing precancers and cancers. An elevated risk represents an above-average risk that a subject will develop cancer, which can be determined, for example, through family history or the detection of genes causing a predisposition to developing cancer.

Compounds described herein include any of their pharmaceutically acceptable forms, including isomers (e.g., diastereomers and enantiomers), tautomers, salts, solvates, polymorphs, prodrugs, and the like. In particular, if a compound is optically active it includes each of the compound's enantiomers as well as racemic mixtures of the enantiomers. It should be understood that the term “compound” includes any or all of such forms, whether explicitly stated or not (although at times, “salts” are explicitly stated).

The term “pharmaceutically acceptable” means that the compound or composition is suitable for administration to a subject to achieve the treatments described herein, without unduly deleterious side effects in light of the severity of the disease and necessity of the treatment.

The term “pharmaceutically acceptable salt” refers to salts prepared from pharmaceutically acceptable non-toxic acids or bases including inorganic acids and bases and organic acids and bases. When the compounds described herein are basic, salts may be prepared from pharmaceutically acceptable non-toxic acids including inorganic and organic acids. Suitable pharmaceutically acceptable acid addition salts for the compounds described herein include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds described herein include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

As used herein, the term “effective amount” refers to an amount of a PP2A activator and an amount of a base excision repair (BER) inhibitor (e.g., PARP inhibitor), the combination of which is sufficient to provide a desired effect. For example, a “therapeutically effective amount” provides an amount that is effective to reduce or arrest a disease or disorder such as abnormal cell growth or cell migration in a subject. The result can be a reduction and/or alleviation of the signs, symptoms, or causes of a disease or disorder, or any other desired alteration of a biological system. The effectiveness of treatment may be measured by evaluating a reduction in tumor load or decrease in tumor growth or tumor cell invasion and/or migration in a subject in response to the administration of a combination of a PP2A activator and BER inhibitor. The reduction in tumor load may be represent a direct decrease in mass, or it may be measured in terms of tumor growth delay, which is calculated by subtracting the average time for control tumors to grow over to a certain volume from the time required for treated tumors to grow to the same volume. The decrease in tumor cell metastasis may represent a direct decrease in tumor cell migration, or it may be measured in terms of the delay of tumor cell metastasis. An effective amount or either a PP2A and/or BER inhibitor in any individual case may be determined by one of ordinary skill in the art using routine experimentation.

The term “potentiate” means to enhance or increase the beneficial activity or efficacy of the anticancer agent over that which would be expected from the anticancer agent alone or the potentiating agent alone.

The term “sensitize” means to alter cancer cells or tumor cells in a way that allows for more effective treatment of the associated neoplastic disease with one or more therapeutics or an anticancer agents.

The term “synergistic effect” means the combined effect of two or more anticancer agents or chemotherapy drugs can be greater than the sum of the separate effects of the anticancer agents or chemotherapy drugs alone. For example, the combined effect of a BER inhibitor, such as a PARP inhibitor, and a PP2A activator can be greater than the sum of the separate effects of the PARP inhibitor and PP2A activator alone. The synergistic effect can be determined using the combination index equation (CIE), wherein synergism has a CI<1.

The term “subject,” “individual,” and “patient” are used interchangeably herein to mean a human or other animal, such as farm animals or laboratory animals (e.g., guinea pig or mice) capable of having cell cycle (influenced) determined diseases, either naturally occurring or induced, including but not limited to cancer.

The terms “subject diagnosed with cancer”, “subject having cancer” or “subjects identified with cancer” refers to patient subjects that are identified as having or likely having cancer. Nonlimiting examples of diagnosing a subject with cancer include diagnoses using histological analysis conducted by a board-certified pathologist and diagnostic tests based on molecular approaches.

The term “small molecule” refers to a low molecular weight organic compound, which is by definition not a polymer. The small molecule can bind with high affinity to a biopolymer, such as protein, nucleic acid, or polysaccharide and in some instances alter the activity or function of the biopolymer. The upper molecular weight limit for a small molecule is about 800 Daltons, which allows for the possibility to rapidly diffuse across cell membranes so that they can reach intracellular sites of action. In addition, this molecular weight cutoff can be a condition for oral bioavailability.

The term “wild type” (wt) cell or cell line is used herein, for purposes of the specification and claims, to mean a cell or cell line that retains the characteristics normally associated with that type of cell or cell line for the physiological process or morphological characteristic that is being examined. It is permissible for the cell or cell line to have non-wild type characteristics for physiological process or morphological characteristics that are not being examined as long as they do not appreciably affect the process or characteristic being examined.

Embodiments described herein relate to compositions and methods for treating cancer, and particularly relates to the use of PP2A activators in combination with base excision repair inhibitors, such as PARP inhibitors, and pharmaceutical compositions including the same, to treat cancer in subjects in need thereof. It has been shown that small molecule compounds can bind and activate protein phosphatase 2A (PP2A), a heterotrimeric tumor suppressor frequently inactivated in human cancer. Activators of PP2A can potently inhibit polo-kinase 1 (Plk1), a major regulator of DNA damage response, and induce cell death in vitro and in vivo. Plk1 is both a biomarker and a therapeutic target given that it is specifically overexpressed in cancer cells, including ovarian cancer where its expression correlates with histological grade and poor patient outcome. It was found that PP2A activators exert an anti-cancer effect through the dephosphorylation and degradation of Plk1. Activation of PP2A results in the coordinate downregulation of Plk1 and other key PP2A regulated oncogenic pathways, such as PI3K-AKT and MAPK. A combination PP2A activator and PARP inhibitor treatment was found to synergistically induce cell death and decrease in vivo tumor burden in PARP resistant cancers, thus providing a method to sensitize tumors to PARP inhibitors, and other BER inhibitors, that rely on defective DNA repair machinery. Accordingly, therapeutically effective amounts of PP2A activators can be administered in combination with BER inhibitors, such as PARP inhibitors, to treat cancer in subjects in need thereof.

PP2A Activators

The PP2A activator can be any drug or compound, such as a pharmacologic chemical species, a complex (e.g., a metal complex), peptide agent, fusion protein, or oligonucleotide that activates the phosphatase and/or induces significant conformational changes in the PP2A complex resulting in decreased inhibitory phophorylation at the Y307 residue.

In some embodiments, PP2A activators can include small molecule activators of PP2A. For example, the PP2A activator can include tricyclic neuroleptic compound derivatives capable of inducing conformational changes in the PP2A complex resulting in decreased inhibitory phophorylation at Y307. In certain embodiments, the PP2A activator can include tricyclic neuroleptic compounds devoid of GPCR or monoamine transporter pharmacology.

In some embodiments, a small molecule tricyclic neuroleptic compound derivative PP2A activator for use in the present invention can include compounds of formula (I):

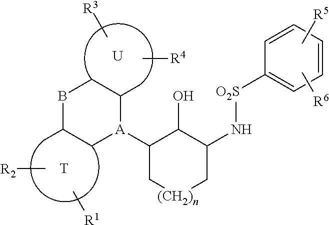

wherein:

B is selected from the group consisting of: direct bond, —O—, —(CH₂—O)—, —(O—CH₂)—, —C(═O)N(CH₃)— and —N(CH₃)C(═O)—;

A is selected from N and CH;

T is a benzene ring or a five or six membered heteroaromatic ring;

U is a benzene ring or a five or six membered heteroaromatic ring;

n is zero, 1 or 2;

R¹, R², R³ and R⁴ are chosen independently from H, OH, halogen, cyano, nitro, (C₁-C₃)alkylamino, (C₁-C₃)dialkylamino, (C₁-C₃)acylamino, (C₁-C₃)alkylsulfonyl, (C₁-C₃)alkylthio, (C₁-C₃)alkyl, (C₁-C₃)haloalkyl, (C₁-C₃)haloalkoxy, —CC(═O)O(C₁-C₃)alkyl, and (C₁-C₃)alkoxy; R⁵ and R⁶ are chosen independently from H, halogen, cyano, nitro, azido, (C₁-C₃)haloalkyl, (C₁-C₃)haloalkoxy, and (C₁-C₃) haloalkylthio.

C₁ to C₂₀ hydrocarbon includes alkyl, cycloalkyl, polycycloalkyl, alkenyl, alkynyl, aryl and combinations thereof. Examples include benzyl, phenethyl, cyclohexylmethyl, adamantyl, camphoryl and naphthylethyl. Hydrocarbyl refers to any substituent comprised of hydrogen and carbon as the only elemental constituents. Aliphatic hydrocarbons are hydrocarbons that are not aromatic; they may be saturated or unsaturated, cyclic, linear or branched. Examples of aliphatic hydrocarbons include isopropyl, 2-butenyl, 2-butynyl, cyclopentyl, norbornyl, etc. Aromatic hydrocarbons include benzene (phenyl), naphthalene (naphthyl), anthracene, etc.

Unless otherwise specified, alkyl (or alkylene) is intended to include linear or branched saturated hydrocarbon structures and combinations thereof. Alkyl refers to alkyl groups from 1 to 20 carbon atoms, preferably 1 to 10 carbon atoms, more preferably 1 to 6 carbon atoms. Examples of alkyl groups include methyl, ethyl, propyl, isopropyl, n-butyl, s-butyl, t-butyl and the like.

Cycloalkyl is a subset of hydrocarbon and includes cyclic hydrocarbon groups of from 3 to 8 carbon atoms. Examples of cycloalkyl groups include cy-propyl, cy-butyl, cy-pentyl, norbornyl and the like.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Heterocycle means an aliphatic or aromatic carbocycle residue in which from one to four carbons is replaced by a heteroatom selected from the group consisting of N, O, and S. The nitrogen and sulfur heteroatoms may optionally be oxidized, and the nitrogen heteroatom may optionally be quaternized. Unless otherwise specified, a heterocycle may be non-aromatic (heteroaliphatic) or aromatic (heteroaryl). Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl.

Alkoxy or alkoxyl refers to groups of from 1 to 20 carbon atoms, preferably 1 to 10 carbon atoms, more preferably 1 to 6 carbon atoms of a straight or branched configuration attached to the parent structure through an oxygen. Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

The term “halogen” means fluorine, chlorine, bromine or iodine atoms. In one embodiment, halogen may be a fluorine or chlorine atom.

Unless otherwise specified, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl and the like. Lower-acyl refers to groups containing one to four carbons. The double bonded oxygen, when referred to as a substituent itself is called “oxo”.

The term “optionally substituted” may be used interchangeably with “unsubstituted or substituted”. The term “substituted” refers to the replacement of one or more hydrogen atoms in a specified group with a specified radical. For example, substituted alkyl, aryl, cycloalkyl, heterocyclyl etc. refer to alkyl, aryl, cycloalkyl, or heterocyclyl wherein one or more H atoms in each residue are replaced with halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino [HNC(═O)O-alkyl], aminocarbonyl (also known as carboxamido) [—C(═O)NH₂], alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkylsulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. “Oxo” is also included among the substituents referred to in “optionally substituted”; it will be appreciated by persons of skill in the art that, because oxo is a divalent radical, there are circumstances in which it will not be appropriate as a substituent (e.g., on phenyl). In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine. In preferred embodiments, substituents are halogen, haloalkyl, alkyl, acyl, hydroxyalkyl, hydroxy, alkoxy, haloalkoxy, aminocarbonyl oxaalkyl, carboxy, cyano, acetoxy, nitro, amino, alkylamino, dialkylamino, alkylthio, alkylsulfinyl, alkylsulfonyl, alkylsulfonylamino arylsulfonyl, arylsulfonylamino, and benzyloxy.

In some embodiments, a small molecule PP2A activator can include compounds of formula (II):

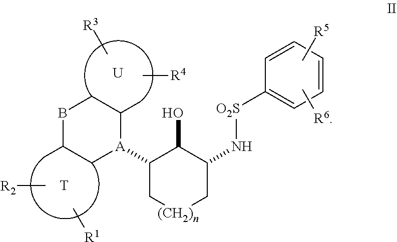

In some embodiments, a small molecule PP2A activator can include compounds of formula (IIIa) or (IIIb):

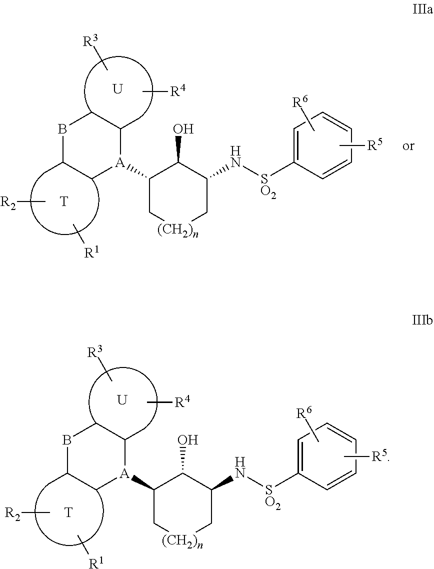

In the embodiments described below, the compound may be of formula I, II, Ina or Mb, unless otherwise indicated.

In some embodiments, n is one. These compounds may be envisioned as N-arylsulfonyl derivatives of 2-aminocyclohexanol:

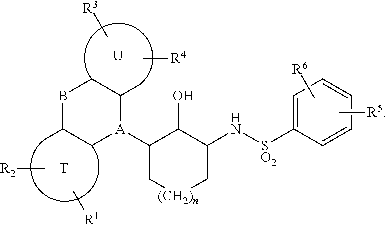

In some embodiments, n is zero. These compounds may be envisioned as N-arylsulfonyl derivatives of 2-aminocyclopentanol:

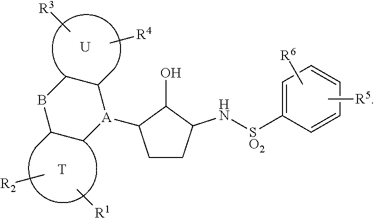

In some embodiments, n is two. These compounds may be envisioned as N-arylsulfonyl derivatives of 2-aminocycloheptanol:

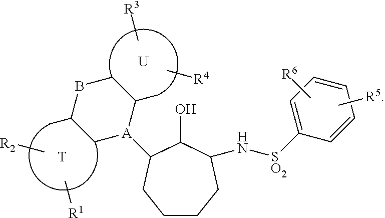

In any of the foregoing subgenera (cyclohexanol, cyclopentanol or cycloheptanol), preferred cycloalkanols are those in which the relative configurations are such that the amine and the tricycle are both trans to the alcohol:

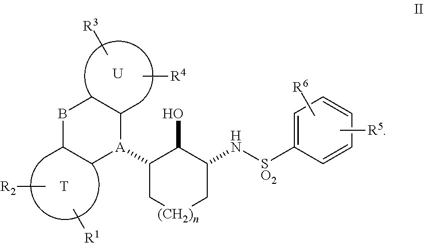

In this trans: trans subgroup, compounds can be either single enantiomers IIIc and IIIb or a mixture of the two. If a mixture, the mixture will most commonly be racemic, but it need not be. Substantially pure single enantiomers of biologically active compounds such as those described herein often exhibit advantages over their racemic mixture.

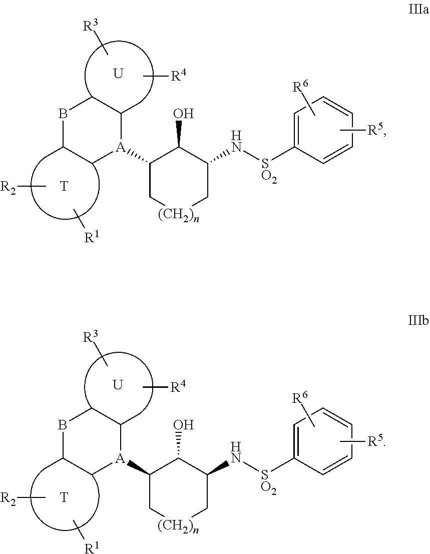

In any of the foregoing subgenera (cyclohexanol, cyclopentanol or cycloheptanol), A may be N or CH. In both the N-series and the CH series, B may be a direct bond, —O—, —(CH₂—O)—, —(O—CH₂)—, —C(═O)N(CH₃)— or —N(CH₃)C(═O)—.

In some embodiments, at least one of T and U is a heterocycle such as pyridine, pyrimidine, diazine, thiophene, thiazole, oxazole, imidazole, pyrrole, or furan. In some embodiments, one of T and U is a benzene ring, and the other of T and U is selected from pyridine, pyrimidine, and thiophene. In other embodiments, T and U are both benzene rings.

When B is a direct bond, T and U are benzene rings and A is N, a subgenus of cycloalkanols in which the tricyclic substituent is a carbazole results:

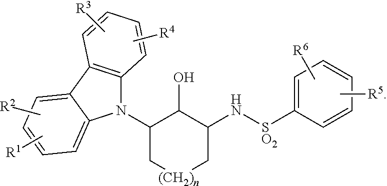

When B is —O—, T and U are benzene rings and A is N, a subgenus of cycloalkanols in which the tricycle is a dibenzooxazine results:

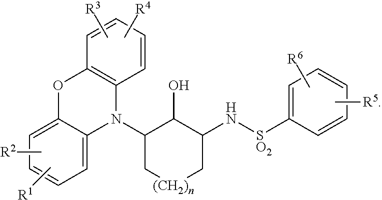

When B is —(CH₂—O)— or —(O—CH₂)—, T and U are benzene rings and A is N, two subgenera of cycloalkanols in which the tricyclic substituent is a dibenzooxazepine result:

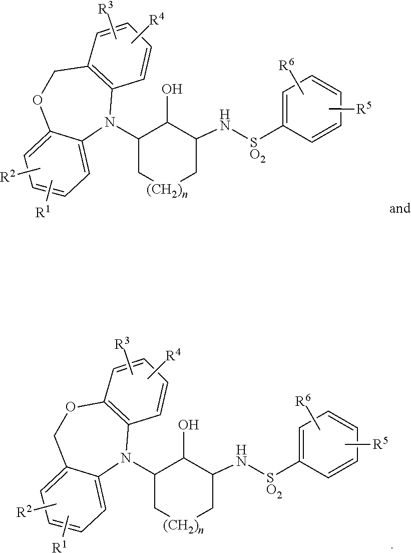

When B is —C(═O)N(CH₃)— or —N(CH₃)C(═O)—, T and U are benzene rings and A is N, two subgenera of cycloalkanols in which the tricyclic substituent is a dibenzodiazepine result:

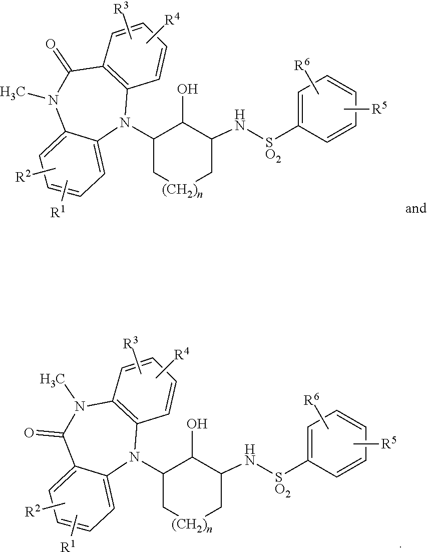

When B is a direct bond, T and U are benzene rings and A is CH, a subgenus of cycloalkanols in which the tricyclic substituent is a fluorene results:

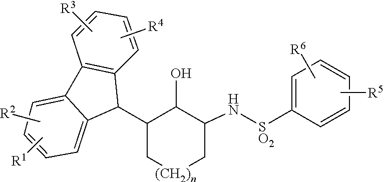

In some embodiments, R² and R⁴ are H, and R¹ and R³ are chosen independently from H, OH, F, Cl, Br, CN, CO₂CH₃, CH₃, CF₃, OCF₃, and OCH₃. In some embodiments, all of R′, R², R³ and R⁴ are H. In some embodiments, at least one of R′, R², R³ and R⁴ is located at a carbon two positions away from a bridgehead carbon. In some embodiments, R⁵ is H, and R⁶ is chosen from H, F, Cl, CF₃, OCF₃, SCF₃, N₃ and —CN. Often R⁶ is in the para position.

Exemplary PP2A activators described herein can be selected from the group consisting of:

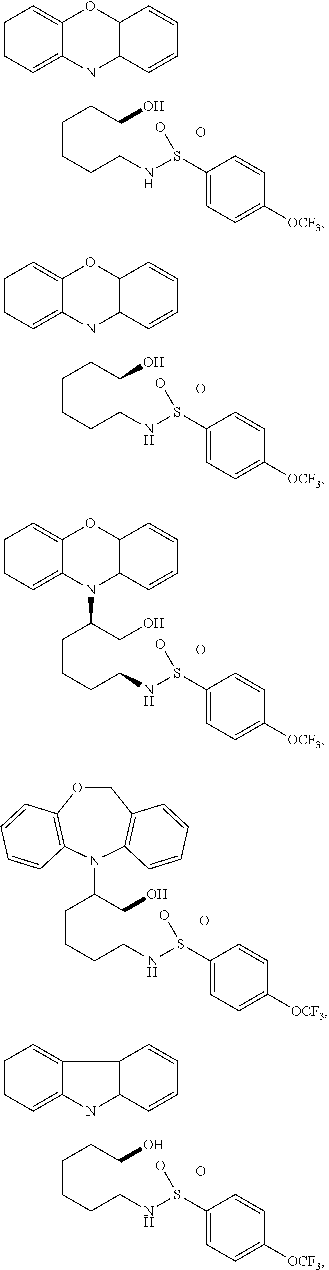

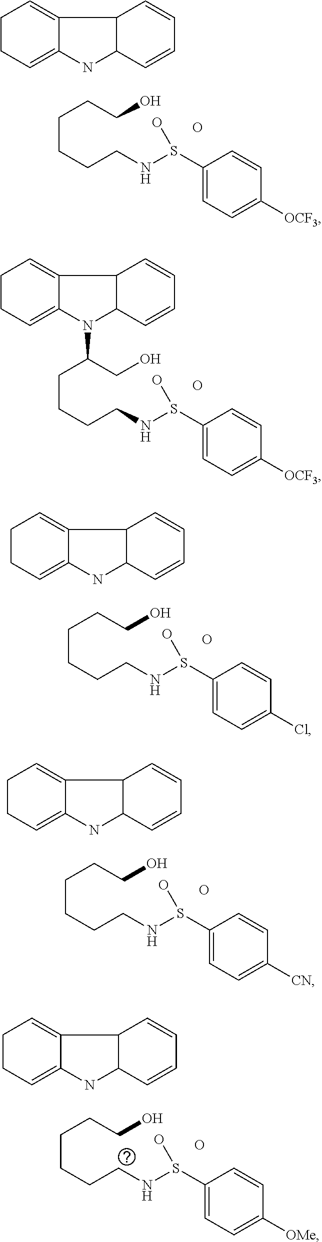

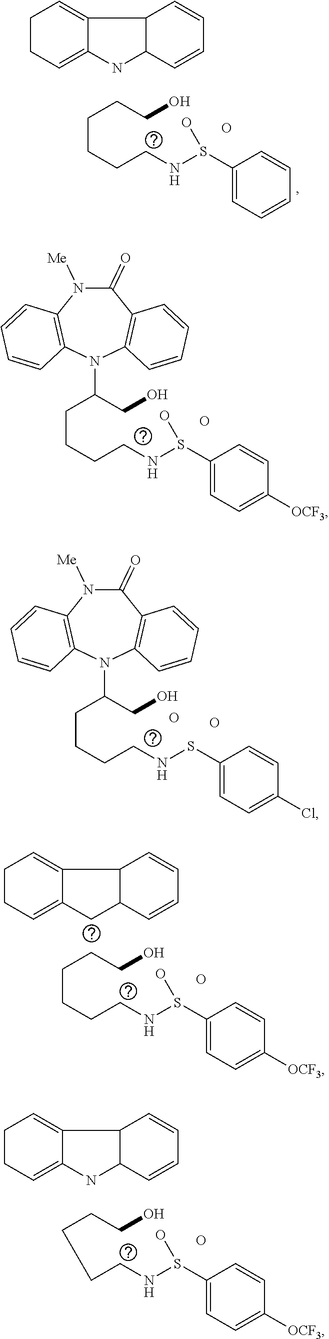

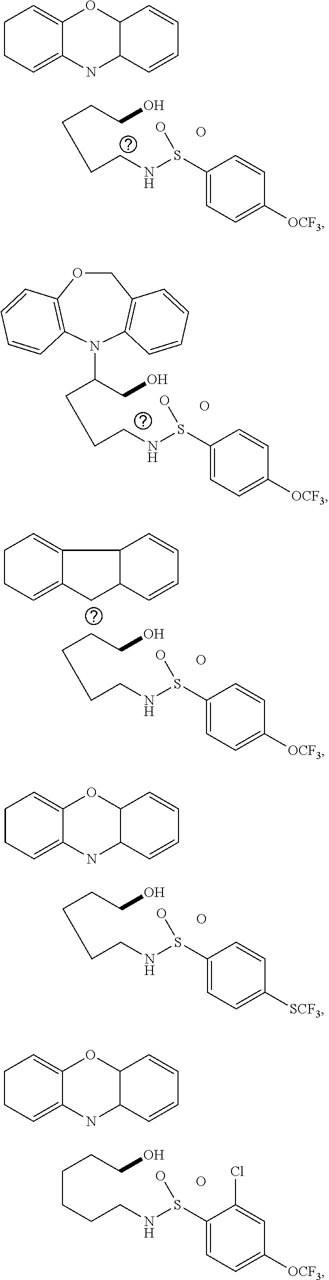

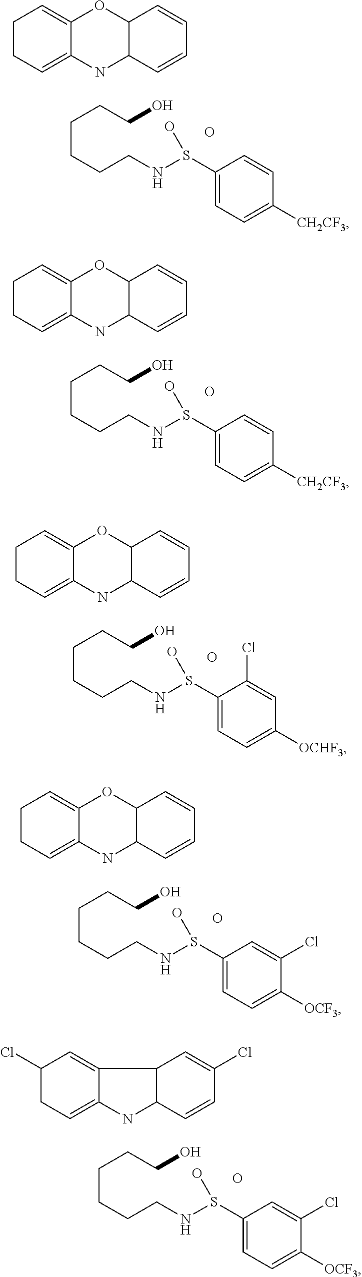

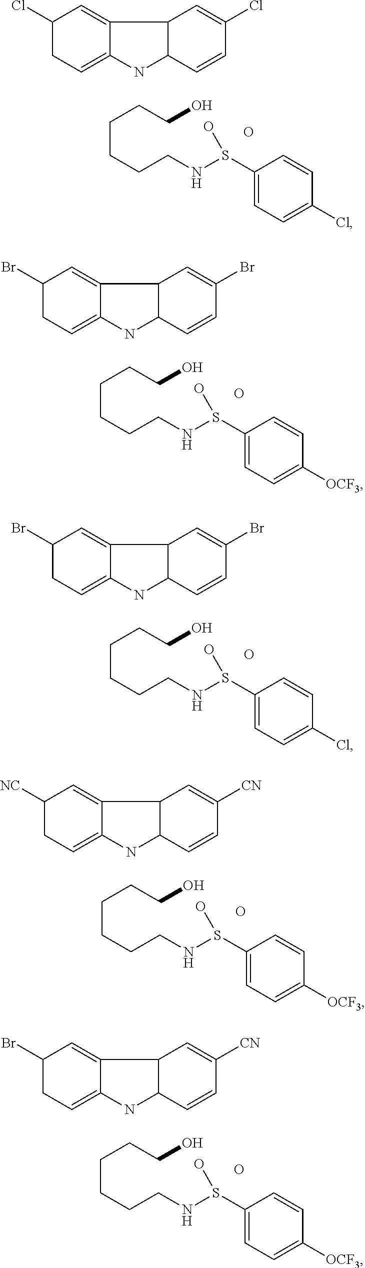

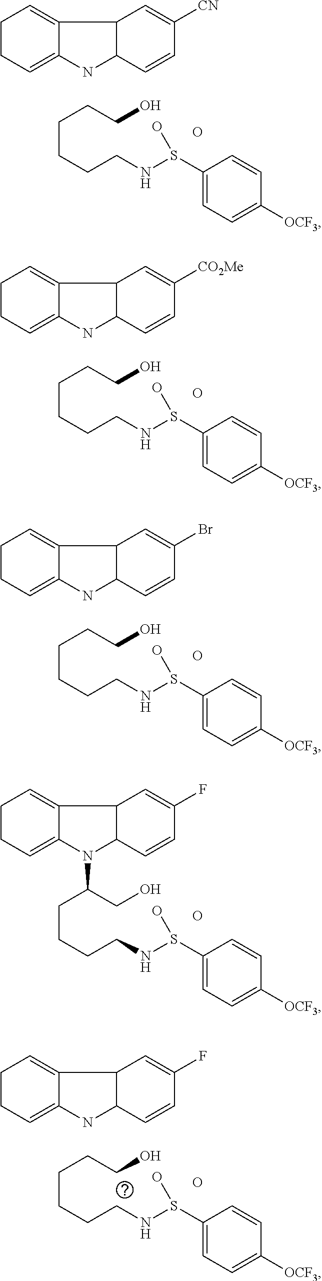

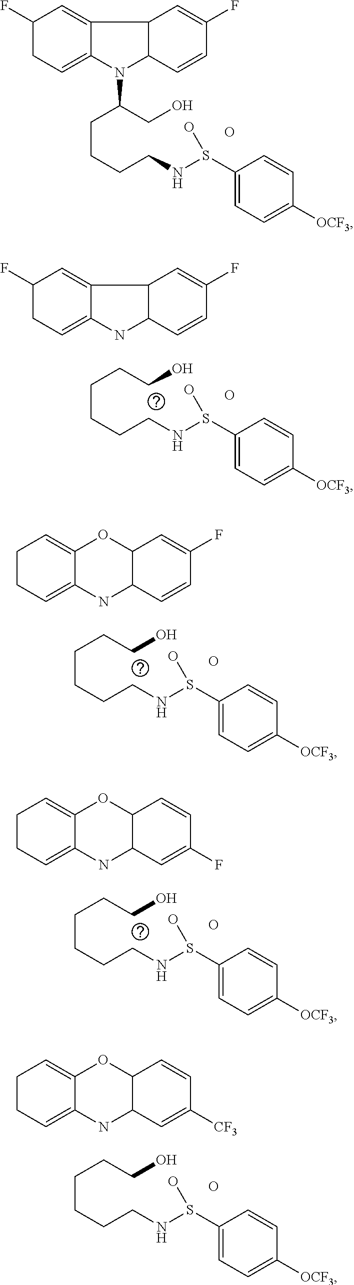

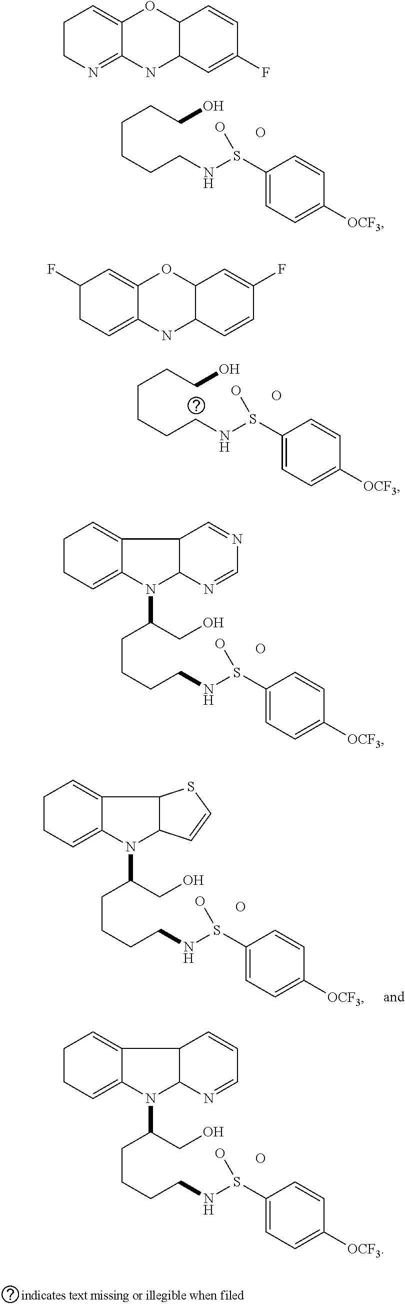

In some embodiments, a small molecule tricyclic neuroleptic compound derivative PP2A activator for use in the present invention can include compounds of formula (IV):

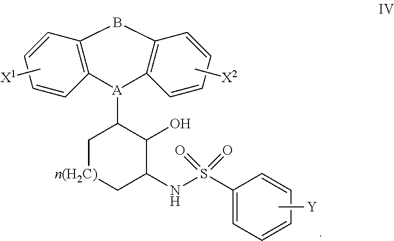

wherein:

B is selected from the group consisting of: —S—, —(CH₂—CH₂)—, and —CH═CH—;

A is selected from N and CH;

n is zero, 1 or 2;

X¹ is selected from —H, —F, —Cl, —CF₃, and —CN;

X² is selected from —H, —F, —Cl, —CF₃, and —CN; and

Y represents one or two substituents each independently selected from —H, —F, —Cl, —(C₁-C₃)haloalkyl, —(C₁-C₃)haloalkoxy, —(C₁-C₃)alkoxy, —C(═O)(C₁-C₃)alkyl, —C(═O)H, —(C₁-C₃)hydroxyalkyl, —(C₁-C₃)haloalkylthio, —N₃, and —CN.

In some embodiments, the invention relates to compounds of formula (V), wherein the relative configurations are such that the amine and the tricycle are both trans to the alcohol:

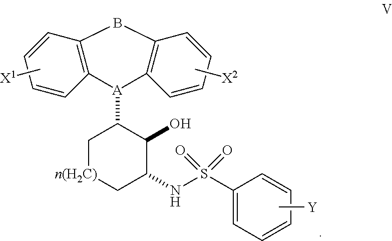

In this trans: trans subgroup, compounds can be either single enantiomers VIa and VIb or a mixture of the two. If a mixture, the mixture will most commonly be racemic, but it need not be. Substantially pure single enantiomers of biologically active compounds such as those described herein often exhibit advantages over their racemic mixture.

In some embodiments, the PP2A activator can include a compound of formula (VIa):

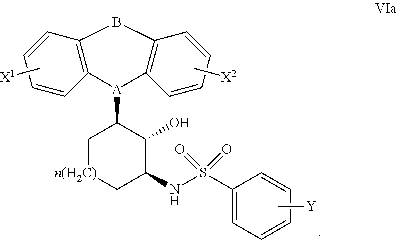

In some embodiments, the PP2A activator can include a compound of formula (VIb):

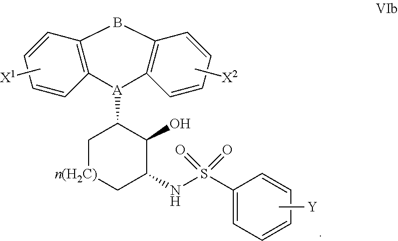

In the embodiments described below, the compound may be of formula IV, V, VIa or VIb, unless otherwise indicated.

In some embodiments, B is —(CH₂—CH₂)—. In some embodiments, B is —S—. In some embodiments, B is —CH═CH—.

In some embodiments, A is N. In some embodiments, A is CH.

In some embodiments, n is zero. In some embodiments, n is one. In some embodiments, n is two.

In some embodiments, X¹ is —H. In some embodiments, X¹ is —F. In some embodiments, X¹ is —Cl. In some embodiments, X¹ is —CF₃. In some embodiments, X¹ is —CN.

In some embodiments, X² is —H. In some embodiments, X² is —F. In some embodiments, X² is —Cl. In some embodiments, X² is —CF₃. In some embodiments, X² is —CN.

In some embodiments, X¹ and X² are both —H.

In some embodiments, Y is —H. In some embodiments, Y is —F. In some embodiments, Y is —Cl. In some embodiments, Y is —(C₁-C₃)haloalkyl. In some embodiments, Y is —CF₃. In some embodiments, Y is —CH₂CF₃ or —CF₂CF₃. In some embodiments, Y is —(C₁-C₃)haloalkoxy. In some embodiments, Y is —OCF₃. In some embodiments, Y is —OCHF₂. In some embodiments, Y is —(C₁-C₃)alkoxy. In some embodiments, Y is —OCH₃. In some embodiments, Y is —C(═O)(C₁-C₃)alkyl. In some embodiments, Y is —C(═O)CH₃. In some embodiments, Y is —C(═O)H. In some embodiments, Y is —(C₁-C₃)hydroxyalkyl. In some embodiments, Y is —C(CH₃)₂OH. In some embodiments, Y is —(C₁-C₃)haloalkylthio. In some embodiments, Y is —SCF₃. In some embodiments, Y is —N₃. In some embodiments, Y is —CN. In some embodiments, one instance of Y is H or Cl, and another instance of Y is selected from —H, —F, —Cl, —(C₁-C₃)haloalkyl, —(C₁-C₃)haloalkoxy, —(C₁-C₃)alkoxy, —C(═O)(C₁-C₃)alkyl, —C(═O)H, —(C₁-C₃)hydroxyalkyl, —(C₁-C₃)haloalkylthio, —N₃, and —CN. In some embodiments, one instance of Y is Cl, and another instance of Y is —OCF₃.

In some embodiments, B is —(CH₂—CH₂)— and n is one. One such example is shown below:

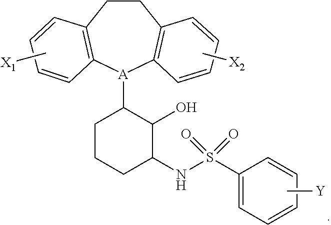

In some embodiments, B is —(CH₂—CH₂)—, A is N, and n is one. One such example is shown below:

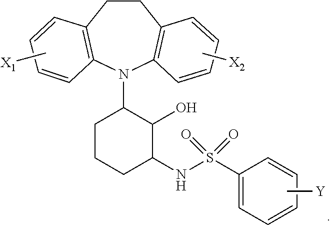

In some embodiments, B is —(CH₂—CH₂)— and A is N. One such example is shown below:

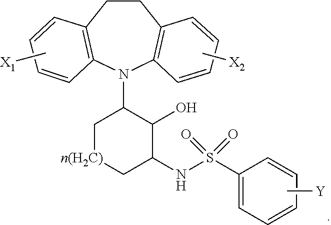

In some of these embodiments, X¹ and X² are both —H. In some embodiments, Y is in the para position, as shown below:

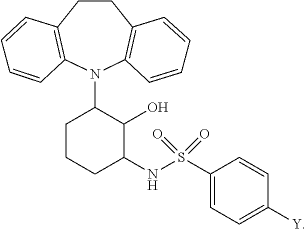

In some embodiments, Y is selected from —H, —F, —Cl, —(C₁-C₃)haloalkyl, —(C₁-C₃)haloalkoxy, —(C₁-C₃)alkoxy, —C(═O)(C₁-C₃)alkyl, —C(═O)H, —(C₁-C₃)hydroxyalkyl, —(C₁-C₃)haloalkylthio, —N₃, and —CN. In some embodiments, Y is selected from —H, —F, —Cl, —CF₃, —CH₂CF₃, —CF₂CF₃—OCF₃, —OCHF₂, —OCH₃, —C(═O)CH₃, —C(═O)H, —C(CH₃)₂OH, —SCF₃, —N₃, and CN. In some embodiments, Y is —OCF₃.

The configuration of any carbon-carbon double bond appearing herein is selected for convenience only and is not intended to designate a particular configuration; thus a carbon-carbon double bond depicted arbitrarily herein as trans may be cis, trans, or a mixture of the two in any proportion. Unless otherwise stated, all tautomeric forms of the compounds of the invention are within the scope of the invention.

The compounds described herein contain one or more asymmetric centers and may thus give rise to enantiomers, diastereomers, and other stereoisomeric forms which may be defined in terms of absolute stereochemistry as (R)- or (S)-. The present invention is meant to include all such possible isomers. Optically active (R)- and (S)-isomers may be prepared using homo-chiral synthons or homo-chiral reagents, or optically resolved using conventional techniques. When the compounds described herein contain olefinic double bonds or other centers of geometric asymmetry, and unless specified otherwise, it is intended to include both (E)- and (Z)-geometric isomers. Likewise, all tautomeric forms are intended to be included.

The graphic representations of racemic, ambiscalemic and scalemic or enantiomerically pure compounds used herein are a modified version of the denotations taken from Maehr J. Chem. Ed. 62, 114-120 (1985): simple lines provide no information about stereochemistry and convey only connectivity; solid and broken wedges are used to denote the absolute configuration of a chiral element; wavy lines indicate disavowal of any stereochemical implication which the bond it represents could generate; solid and broken bold lines are geometric descriptors indicating the relative configuration shown but not necessarily denoting racemic character; and wedge outlines and dotted or broken lines denote enantiomerically pure compounds of indeterminate absolute configuration. For example, the graphic representation

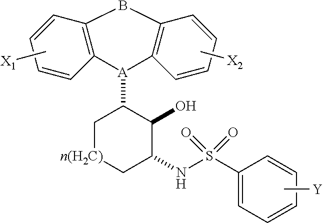

indicates either, or both, of the two trans: trans enantiomers:

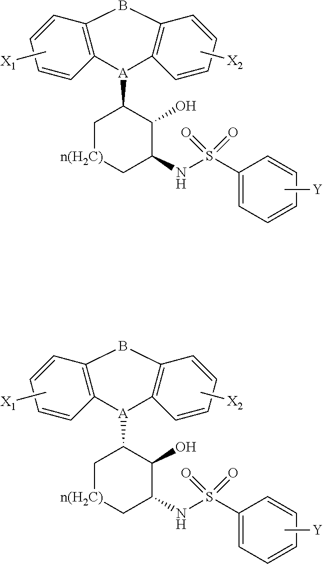

in any ratio, from pure enantiomers to racemates. The graphic representation:

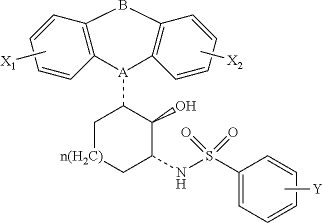

indicates a single enantiomer of unknown absolute stereochemistry, i.e., it could be either of the two preceding structures, as a substantially pure single enantiomer. And, finally, the representation:

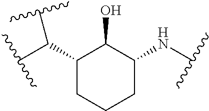 [SMILES: *N[C@@H]1CCC[C@H](C(*)*)[C@H]1O]

indicates a pure (1R,2R,6S)-2-amino-6-(C-attached tricycle)cyclohexanol. For the purpose of the present disclosure, a “pure” or “substantially pure” enantiomer is intended to mean that the enantiomer is at least 95% of the configuration shown and 5% or less of other enantiomers. Similarly, a “pure” or “substantially pure” diastereomer is intended to mean that the diastereomer is at least 95% of the relative configuration shown and 5% or less of other diastereomers. In the text describing the stereochemistry of the examples, the convention of Chemical Abstracts is used. Thus “(1R,2R,6S)-rel-” indicates that the three chiral centers are in that relative relationship, which would be depicted in a structural diagram by solid bold and dashed lines, whereas “(1R,2R,6S)” without the “rel” indicates a single enantiomer of that absolute configuration, which would be depicted in a structural diagram by solid and broken wedges.

In an exemplary embodiment, the PP2A activator can be selected from the group consisting of:

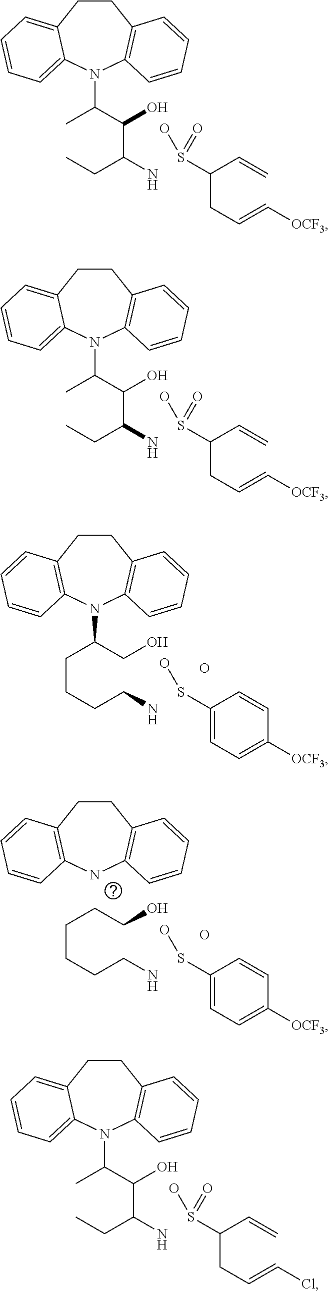

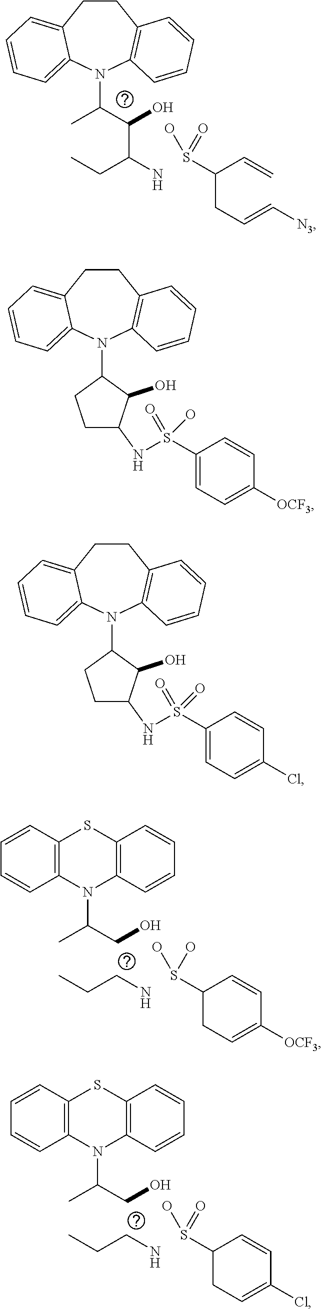

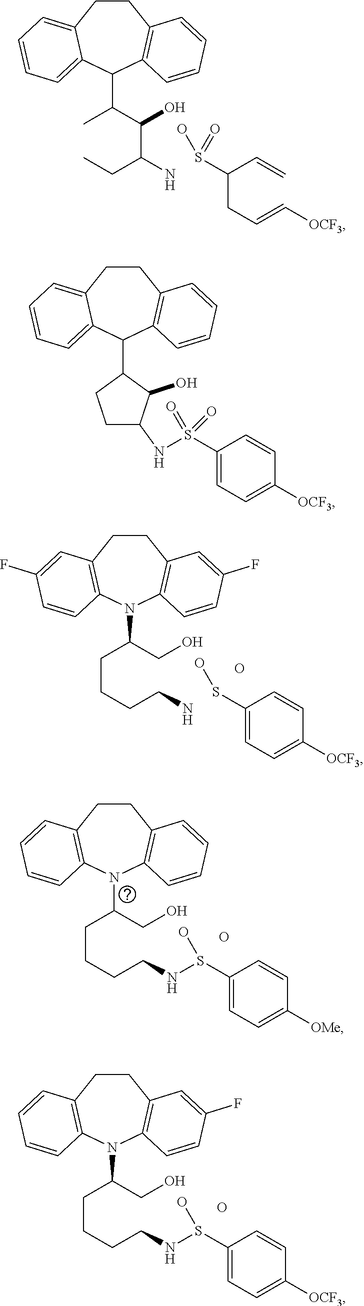

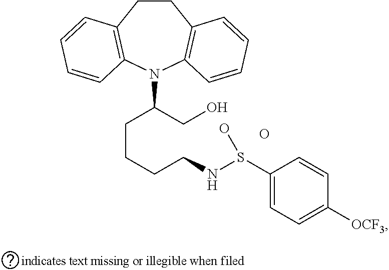

Additional agents capable of activating the PP2A phosphatase for use in methods described herein may be selected from the group consisting of, but not limited to, FTY720 (also called fingolimod), forskolin, 1,9-dideoxyforskolin, ceramides (also called sphingosines), such as C2-ceramide, topoisomerase inhibitors, such as etoposide (Eposin, Etopophos, Vepesid™, VP-16™), tubulin polymerisers, such as methyl-3,5-diiodo-4-(4′-methoxypropoxy)benzoate (DIME or DIPE), fatty acids, such as palmitate, and thiol alkylating agents such as N-ethylmaleimide (NEM).

Further agents for increasing PP2A activity for prophylaxis or treatment of cancers as described herein include genetic molecules, such as over expression constructs for the endogenous PP2A activator PTPA, PP2A or individual PP2A gene subunits. Similarly, such agents may also take the form of DNA/RNA inhibition molecules, such as shRNA or antisense sequences, including those specific to the endogenous PP2A inhibitor SET, or to an individual PP2A gene subunit or specific region of the PP2A gene (e.g., a transcriptional regulatory control subunit such as a promoter).

Candidate PP2A activators or activating agents may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al, Proc. Natl. Acad. Sci. USA, 92:3439-3443 (1995)). Candidate agents can be used in these animal models to determine if a candidate agent activates PP2A activity, decreases one or more of the symptoms associated with the cancer, including, for instance, cancer metastasis, cancer cell motility, cancer cell invasiveness, or combinations thereof.

Base Excision Repair Inhibitors

Base excision repair (BER) is initiated by a DNA glycosylase that removes N-glycosidic (base-sugar) bonds, liberating the damaged base and generating an abasic site (e.g., an apurinic or apyrimidinic (AP) site). An apurinic or apyrimidinic (AP) site results from the loss of a purine or pyrimidine residue, respectively, from DNA (deoxyribonucleic acid). Uracil residues can form from the spontaneous deamination of cytosine and can lead to a C→T transition if unrepaired. There is also a glycosylase that recognizes and excises hypoxanthine, the deamination product of adenine. Other glycosylases remove alkylated bases (such as 3-methyladenine, 3-methylguanine, and 7-methylguanine), ring-opened purines, oxidatively damaged bases, and in some organisms, UV photodimers.

The AP site is further processed by a 5′-3′ endonuclease (AP endonuclease (APE)) that incises the phosphodiester bond on both sides of the damaged purine or pyrimidine base. The AP endonucleases introduce chain breaks by cleaving the phosphodiester bonds at the AP sites.

PARP aids in processing of DNA strand breaks induced during BER. PARP is a DNA nick surveillance protein that binds weakly to BER intermediates when single-nucleotide BER proceeds normally to completion. In contrast, when single nucleotide BER is stalled by a block in the excision step, PARP binds strongly to the BER intermediate, along with AP endonuclease (APE), DNA pol β, and 1-BN-1.

In mammalian cells, the 5′-deoxyribose sugar phosphate is removed by the intrinsic AP lyase (dRP) activity of DNA polymerase β (pol β). DNA polymerase enzyme also fills the gaps with new nucleotides.

Finally, DNA ligase covalently links the 3′ end of the new material to the old material. Thus, the wild-type sequence is restored.

Topoisomerases I and II are also involved in DNA repair, as they recognize spontaneous AP sites and form stable cleavable complexes. Topoisomerase II inhibitors promote DNA cleavage and other chromosomal aberrations, including sister chromatid exchanges.

In some embodiments, the BER inhibitor that is administered in combination with the PP2A activator is a Poly [ADP-Ribose] Polymerase (PARP1) inhibitor. PARP inhibitors that can be administered in combination with the PP2A include, but are not limited to, nicotinamide; NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1(2H)-pyridinyl)propyl]-4(3H)-quinazo-linone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5[4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281); ABT-888 (Veliparib); BSI-201 (Iniparib); Rucaparib (AG-014699); INO-1001; A-966492; PJ-34; and PARP1 inhibitors described in U.S. patent application Ser. No. 12/576,410, which is incorporated by reference in its entirety.

Other examples of BER inhibitors that can be administered to the subject in combination with the PP2A activator and optionally the PARP inhibitor include AP endonuclease inhibitors, such as methoxyamine (MX) or salts thereof, DNA polymerase inhibitors (e.g., DNA polymerase β, γ or ε), such as prunasin, aphidicolin, 2′,3′-dideoxycytidine triphosphate (ddCTP), 2′,3′-dideoxythymidine triphosphate (ddTTP), 2′,3′-dideoxyadenosine triphosphate (ddATP), 2′,3′-dideoxyguanosine triphosphate (ddGTP), 1-beta-D-arabinofuranosylcytosine (Ara-C), arabinocytidine, and bleomycin.

Still other examples of BER inhibitors that can be administered to the subject in combination with the PP2A activator and optionally the PARP inhibitor include DNA ligase inhibitors (e.g., DNA ligase I, II, or III), such as ursolic and oleanolic acids, aleuritolic acid, protolichesterinic acid, swertifrancheside, fulvoplumierin, fagaronine chloride, and bleomycin. XRCC1 is the protein partner of DNA ligase III, and inhibitors of XRCC1, such as 3-AB, are useful as BER inhibitors as well.

Further examples of BER inhibitors that can be administered to the subject in combination with the PP2A activator and optionally the PARP inhibitor include topoisomerase II inhibitors. Topoisomerase inhibitors induce DNA cleavage and other chromosomal aberrations, including sister chromatid exchanges. Compounds useful as BER inhibitors also include topoisomerase II inhibitors, such as etoposide (VP-16, VP-16-123), meso-4,4′-(2,3-butanediyl)-bis-(2,6-piperazinedione) (ICRF-193, a bisdioxopiperazine), doxorubicin (DOX), L amsacrine (4′,9-acridinylaminomethanesulfon-m-anisidide; mAMSA), pazelliptine, nalidixic acid, oxolinic acid, novobiocin, coumermycin A1, fostriecin, teniposide, mitoxantrone, daunorubicin, N-[12-dimethylamino)ethyl]acridine-4-carboxamide (DACA), merbarone, quinacrine, ellipticines, epipodophyllotoxins, ethidium bromide, epirubicin, pirarubicin, 3′-deamino-3′-morpholino-13-deoxo-10-hydroxy caminomycin; 2″,3″-bis pentafluorophenoxyacetyl-4′,6′-ethylidene-beta-D glucoside of 4′-phosphate-4′-dimethylepipodophyollotoxin 2N-methyl glucamine salt (F11782; a fluorinated lipophilic epipodophylloid), adriamycin, actinomycin D, anthracyclines (such as 9-aminoanthracycline), and pyrazoloacridine (WA). Topoisomerase I inhibitors, such as camptothecin and topotecan can also be used as BER inhibitors.

In some embodiments, other enzyme inhibitors, whether known in the art or hereafter identified, as well as inhibitors of other elements of the BER pathway, such as DNA alkyltransferase, may be employed in compositions and methods without departing from the scope and spirit of the present embodiments.

In still other embodiments, the PP2A activator and the BER inhibitor, such as a PARP inhibitor, can be administered to the subject in combination with protein kinase inhibitor to further activate PP2A.

In some embodiment, the kinase inhibitor administered in combination with a PP2A activator and BER inhibitor is an IKK inhibitor. IKKs and related kinases positively regulate NF-κB by phosphorylating its inactive cytoplasmic complex with IκB to release NF-κB which translocates to the cell nucleus where it is transcriptionally active. NF-κB is a transcription factor whose dysregulation and overactivation has been implicated in the pathogenesis of many cancers, for example malignant melanoma. (see for example D. Melisi and P. Chaio, NF-kB as a target for cancer therapy in Expert Opin. Ther. Targets (2007) 11(2):133-144 and Michael Karin et al., THE IKK NF-κB SYSTEM: A TREASURE TROVE FOR DRUG DEVELOPMENT, Nature Reviews Drug Discovery Volume 3 2004 17-26). PP2A negatively regulates NF-κB, for example by dephosphorylation of its Rel-A subunit, see J. Yang et al., Protein Phosphatase 2A Interacts with and Directly Dephosphorylates RelA, Vol. 276, No. 51, December 21, pp. 47828-47833, 2001 and X. Lu and W. Yarbrough, Negative regulation of RelA phosphorylation: Emerging players and their roles in cancer, Cytokine & Growth Factor Reviews 26 (2015) 7-13.

Several IKK inhibitors have been developed to suppress or inhibit NF-κB function, for example, N-(6-chloro-9H-pyrido[3,4-b]indol-8-yl)nicotinamide [PS-1145]; N¹-(1,8-dimethylimidazo[1,2-a]quinoxalin-4-yl)ethane-1,2-diamine [BMS-345541]; 1-((5-methoxy-2-(thiophen-2-yl)quinazolin-4-yl)amino)-3-methyl-1H-pyrrole-2,5-dione [SPC-839]; N-(6-chloro-7-methoxy-9H-pyrido[3,4-b]indol-8-yl)-2-methylnicotinamide [ML120B]; 4-amino-[2,3′-bithiophene]-5-carboxamide [SC-514]; (E)-1-(6-(4-chlorophenoxy)hexyl)-2-cyano-3-(pyridin-4-yl)guanidine [CHS828 (GMX1778)]; and (Z)-3-(2,4-dimethyl-5-((2-oxoindolin-3-ylidene)methyl)-1H-pyrrol-3-yl)propanoic acid [SU6668] as anticancer agents, see D. Lee and M. Hung, Advances in Targeting IKK and IKK-Related Kinases for Cancer Therapy, in Clin Cancer Res 2008; 14(18) Sep. 15, 2008. Coadministration of PP2A activating compounds with IKK inhibitors can therefore increase the effectiveness of either agent as an anticancer therapy.

Non-limiting examples of IKK kinase inhibitors include N-(6-chloro-9H-pyrido[3,4-b]indol-8-yl)nicotinamide; N¹-(1,8-dimethylimidazo[1,2-a]quinoxalin-4-yl)ethane-1,2-diamine; 1-((5-methoxy-2-(thiophen-2-yl)quinazolin-4-yl)amino)-3-methyl-1H-pyrrole-2,5-dione; N-(6-chloro-7-methoxy-9H-pyrido[3,4-b]indol-8-yl)-2-methylnicotinamide; 4-amino-[2,3′-bithiophene]-5-carboxamide; (E)-1-(6-(4-chlorophenoxy)hexyl)-2-cyano-3-(pyridin-4-yl)guanidine; and (Z)-3-(2,4-dimethyl-5-((2-oxoindolin-3-ylidene)methyl)-1H-pyrrol-3-yl)propanoic acid.

In other embodiment, the kinase inhibitor administered in combination with a PP2A activator and BER inhibitor is an src or Jak2 kinase inhibitor. PP2A is subject to several levels of regulation including post translation modification by phosphorylation, for example see Maud Martin et al., Recent insights into Protein Phosphatase 2A structure and regulation: the reasons why PP2A is no longer considered as a lazy passive housekeeping enzyme in Biotechnol. Agron. Soc. Environ. 2010 14(1), 243-252 and V Jannsens et al. in PP2A holoenzyme assembly: in cauda venenum (the sting is in the tail) in Trends in Biochemical Sciences Vol. 33 (2008) No. 3, 113-121. Thus phosphorylation on tyrosine-307 of the catalytic subunit serves to inhibit or diminish phosphatase activity. Among the kinases known to phosphorylate tyrosine-307 of the PP2A catalytic subunit is Src (others are lck and Jak2), and several src kinase inhibitors have been developed as anti-cancer agents in their own right, see for example L Kim et al, Src kinases as therapeutic targets for cancer, Nat. Rev. Clin. Oncol. 6, 587-595 (2009).

In certain embodiments, the protein kinase inhibitor is a Jak 2 inhibitor. Non-limiting examples of Jak 2 inhibitors include ruxolitinib, Baricitinib, CYT387, lestaurtinib, pacritinib and TG101348.

In still other embodiments, the protein kinase inhibitor can be a Chk1 kinase inhibitor. PP2A can interact with endogeneous inhibitor proteins, such as CIP2A. Decreased expression of inhibitor proteins, such as CIP2A, promotes PP2A activity. Chk1 kinase inhibitors have been reported as anticancer agents in their own right and furthermore Chk1 kinase inhibition has been shown to decrease CIP2A expression and promote PP2A activity, see A. Khanna et al, Chk1 Targeting Reactivates PP2A Tumor Suppressor Activity in Cancer Cells, Cancer Res; 73(22) Nov. 15, 2013. Thus, coadministration of PP2A activators described above can increase the effectiveness of Chk1 kinase inhibitors as an anticancer therapy.

Examples of Chk1 kinase inhibitors include (S)-5-(3-fluorophenyl)-N-(piperidin-3-yl)-3-ureidothiophene-2-carboxamide [AZD-7762]; (S)-1-(5-bromo-4-methyl-2-(morpholin-2-ylmethoxy)phenyl)-3-(5-methylpyrazin-2-yl)urea [LY2603618 (Rabusertib)]; 6-bromo-3-(1-methyl-1H-pyrazol-4-yl)-5-(piperidin-3-yl)pyrazolo[1,5-a]pyrimidin-7-amine [MK8776 (Sch900776)]; (S)-3-(1H-benzo[d]imidazol-2-yl)-6-chloro-4-(quinuclidin-3-ylamino)quinolin-2(1H)-one [CHIR-124]; and (R)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide [PF-477736].

In other embodiments, the protein kinase inhibitor can be a GSK-3 inhibitor. GSK-3 is a protein kinase whose dysregulation and over activation has been implicated in the pathology of several diseases including cancer (see for example: J. McCubrey et al. in “GSK-3 as potential target for therapeutic intervention in cancer”, Oncotarget, volume 5, number 10, 2881-2911(2014); and A. Martinez et al. in “Glycogen Synthase Kinase 3 (GSK-3) Inhibitors as New Promising Drugs for Diabetes, Neurodegeneration, Cancer, and Inflammation” in Medicinal Research Reviews, Vol. 22, No. 4, 373-384, 2002). Several inhibitors of GSK3 and its isoforms have been developed and proposed as treatments for these conditions as reported in P. Cohen and M. Goedert, Nature Reviews Drug Discovery, volume 3, 479-487 (2004). 3-((3-chloro-4-hydroxyphenyl)amino)-4-(2-nitrophenyl)-1H-pyrrole-2,5-dione [SB415286]; 3-(2,4-dichlorophenyl)-4-(1-methyl-1H-indol-3-yl)-1H-pyrrole-2,5-dione [SB216763]; 6-((2-((4-(2,4-dichlorophenyl)-5-(4-methyl-1H-imidazol-2-yl)pyrimidin-2-yl)amino)ethyl)amino)nicotinonitrile [CHIR-99021 (CT-99021)]; N²-(2-((4-(2,4-dichlorophenyl)-5-(1H-imidazol-2-yl)pyrimidin-2-yl)amino)ethyl)-5-nitropyridine-2,6-diamine [CHIR-98014]; 1-(quinolin-4-yl)-3-(6-(trifluoromethyl)pyridin-2-yl)urea [A1070722 (AXON 1909)]; 4-benzyl-2-(naphthalen-1-yl)-1,2,4-thiadiazolidine-3,5-dione [Tideglusib (NP-12, NP031112)]; and 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione [LY2090313]. Furthermore, GSK-3b has been found to negatively regulate PP2A by indirectly promoting the inhibitory PP2A tyrosine-307 phosphorylation of its catalytic subunit. Thus, inhibition of GSK-3b decreases PP2A tyrosine-307 phosphorylation in vitro and in vivo and hence activates PP2A. See X. Yao et al. in “Glycogen synthase kinase-3β regulates Tyr 307 phosphorylation of protein phosphatase-2A via protein tyrosine phosphatase 1B but not Src”, Biochem. J. (2011) 437, 335-344. Thus, coadministration of a PP2A activator with a GSK-3 inhibitor will increase the effectiveness of either compound in the treatment of cancer. In some embodiments, the GSK-3 inhibitor is 3-((3-chloro-4-hydroxyphenyl)amino)-4-(2-nitrophenyl)-1H-pyrrole-2,5-dione; 3-(2,4-dichlorophenyl)-4-(1-methyl-1H-indol-3-yl)-1H-pyrrole-2,5-dione; 6-((2-((4-(2,4-dichlorophenyl)-5-(4-methyl-1H-imidazol-2-yl)pyrimidin-2-yl)amino)ethyl)amino)nicotinonitrile; N²-(2-((4-(2,4-dichlorophenyl)-5-(1H-imidazol-2-yl)pyrimidin-2-yl)amino)ethyl)-5-nitropyridine-2,6-diamine; 1-(quinolin-4-yl)-3-(6-(trifluoromethyl)pyridin-2-yl)urea; 4-benzyl-2-(naphthalen-1-yl)-1,2,4-thiadiazolidine-3,5-dione; and 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.

In still other embodiments, the protein kinase inhibitor can be an EGFR inhibitor. Non-limiting examples of an EGFR inhibitor can include erlotinib, gefitinib, lapatinib, and icotinib.

Additional protein kinase inhibitors for use in the treatment of cancer in accordance with methods described can include the small molecules afatinib, apatinib, axitinib, cabozantinib, canertinib, certinib, crenolanib, foretinib, crizotinib, dabrafenib, everolimus, ibrutinib, imatinib, lenvatinib, linifanib, motosanib, nilotinib, nintedanib, palbociclib, pazopanib, ponatinib, radotinib, regorafenib, sirolimus, sorafenib, sunitinib, tofacitinib, temsirolimus, trametinib, vandetanib, vatalanib, vemurafenib, fostamatinib, mubritinib, SU6656, the monoclonal antibodies bevacizumab, cetuximab, panitumumab, ranibizumab, trastuzumab, and the RNA aptamer pegaptanib.

Methods of Treating Cancer

The PP2A activators and BER inhibitors described herein can be used in methods of treating cancer in a subject. The methods can include administering to the subject therapeutically effective amounts of at least one PP2A activator in combination with at least one BER inhibitor described above, or pharmaceutically acceptable salt forms thereof.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression.

In certain embodiments, the cancer that is treated includes Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In other embodiments, the cancer is selected from biliary cancer, breast cancer, colorectal cancer, leukemia, acute myeloid leukemia (AML), acute lymphoblastic leukemia (ALL), chronic lymphocytic leukemia (CLL), chronic myelogenous leukemia (CML), hairy cell leukemia, T-cell leukemia, brain malignancy, lymphoma, diffuse large B-cell lymphoma (DLBCL), follicular lymphoma, Hodgkin's lymphoma, MALT lymphoma, mantle cell lymphoma (MCL), no-Hodgkin lymphoma (NHL), endometrial cancer, head and neck cancers, Kaposi's sarcoma, lung cancer, melanoma, multiple myeloma (MM), myelodisplastic disease (MDS), ocular disease, ovarian cancer, pancreatic cancer, prostate cancer, renal cancer, thyroid cancer, tuberous sclerosis, and Waldenstrom macrogloulinemia (WM).

In some embodiments, the cancer can be characterized by over expression of Plk1. For example, the cancer can be selected from the group consisting of: ovarian, pancreatic, renal cell, breast, prostate, lung, hepatocellular carcinoma, glioma, leukemia, lymphoma, colorectal cancers, and sarcomas that overexpress Plk1.

In other embodiments, the cancer can be resistant to treatment with a BER inhibitor, such as a PARP inhibitor. For example, the cancer can include those that are BRCA1/2 wild type, that is, the subject has a BRCA genotype not associated with an increased risk of hereditary breast-ovarian cancer syndrome.

In other embodiments, the subject or cancer can have a BRCA1/2 mutation, that is, the subject has a BRCA genotype associated with an increased risk of hereditary breast-ovarian cancer syndrome.

In still other embodiments, the cancer treated with the PP2A activator and BER inhibitor can be ovarian cancer or breast cancer

Subjects potentially benefiting from the methods described herein include male and female mammalian subjects, including humans, non-human primates, and non-primate mammals. Other mammalian subjects include domesticated farm animals (e.g., cow, horse, pig) or pets (e.g., dog, cat). In some embodiments, the subject can include any human or animal subject who has a disorder characterized by unwanted, rapid cell proliferation of brain cells. Such disorders include, but are not limited to cancers and precancers, such as those described above. For methods of prevention, the subject can include any human or animal subject, and preferably is a human subject who is at risk of obtaining a disorder characterized by unwanted, rapid cell proliferation, such as cancer. The subject may be at risk due to exposure to carcinogenic agents, being genetically predisposed to disorders characterized by unwanted, rapid cell proliferation, and so on.

In certain embodiments, prior to treatment, the patients are selected for having a particular cancer, or for being at risk of a particular cancer. The presence of cancer can be determined by means well known to clinicians. Initial assessment of cancer is based on symptoms presented by the patient. In addition, there are follow-up diagnostic procedures, including, but not limited to PET scans, CAT scans, biopsies, and bio-marker assessments.

Administration and Formulation Therapeutic Agents

Also provided herein are pharmaceutical compositions for the treatment of cancer comprising a PP2A activator, a BER inhibitor, such as a PARP inhibitor, or a combination of a PP2A activator and a BER inhibitor, or a pharmaceutically acceptable salt form thereof, and a pharmaceutically acceptable carrier or diluent.

While it may be possible for therapeutic compounds described herein to be administered as the raw chemical, it is preferable to present them as a pharmaceutical composition. Pharmaceutical compositions described herein can include a PP2A activator and/or a BER inhibitor, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association one or more therapeutic compounds described above or a pharmaceutically acceptable salt thereof (“active ingredient”) with the carrier which constitutes one or more accessory ingredients. In general, the formulations are prepared by uniformly and intimately bringing into association one or more active ingredients with liquid carriers or finely divided solid carriers or both and then, if necessary, shaping the product into the desired formulation.

Thus, in certain embodiments, formulations are prepared by uniformly and intimately bringing into association a PP2A activator and a BER inhibitor with liquid carriers or finely divided solid carriers or both and then, if necessary, shaping the product into the desired formulation, thereby resulting in a coformulation of a PP2A activator and a BER inhibitor for use in a method described herein.

Formulations that can be used for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

In some embodiments, PP2A activators and BER inhibitors described above can be administered to a subject systemically, (i.e., enteral or parenteral administration). Preparations suitable for oral administration are a solution prepared by dissolving an effective amount of an agent or a pharmaceutically acceptable salt thereof in a diluent such as water, physiological saline, or orange juice; capsules, sachets or tablets comprising an effective amount of one or more therapeutic agents in solid or granular form; a suspension prepared by suspending an effective amount of active ingredient in an appropriate dispersant; an emulsion prepared by dispersing and emulsifying a solution of an effective amount of active ingredient in an appropriate dispersant, and the like.

As preparations suitable for parenteral administration (e.g., intravenous administration, subcutaneous administration, intramuscular administration, topical administration, intraperitoneal administration, intranasal administration, pulmonary administration and the like), aqueous and non-aqueous isotonic sterile injectable liquids are available, which may comprise an antioxidant, a buffer solution, a bacteriostatic agent, an isotonizing agent and the like. Aqueous and non-aqueous sterile suspensions can also be mentioned, which may comprise a suspending agent, a solubilizer, a thickener, a stabilizer, an antiseptic and the like. The preparation can be included in a container such as an ampoule or a vial in a unit dosage volume or in several divided doses. An active ingredient and a pharmaceutically acceptable carrier can also be freeze-dried and stored in a state that may be dissolved or suspended in an appropriate sterile vehicle just before use. In addition to liquid injections, inhalants and ointments are also acceptable. In the case of an inhalant, an active ingredient in a freeze-dried state is micronized and administered by inhalation using an appropriate inhalation device. An inhalant can be formulated as appropriate with a conventionally used surfactant, oil, seasoning, cyclodextrin or derivative thereof and the like as required. In some embodiments, the PP2A activators and/or BER inhibitors may be incorporated into sustained-release preparations and devices.

The dosage of the PP2A activators and/or BER inhibitors administered to the subject can vary depending on the kind and activity of active ingredient(s), seriousness of disease, animal species being the subject of administration, drug tolerability of the subject of administration, body weight, age and the like, and the usual dosage, based on the amount of active ingredient per day for an adult, can be about 0.0001 to about 100 mg/kg, for example, about 0.0001 to about 10 mg/kg, preferably about 0.005 to about 1 mg/kg. In certain embodiments, dosage can be about 10 mg/kg. The daily dosage can be administered, for example in regimens typical of 1-4 individual administration daily. Other preferred methods of administration include intraarticular administration of about 0.01 mg to about 100 mg per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

In another embodiment, the practice of the method in conjunction with additional therapies is contemplated. Additional therapies can include conventional chemotherapy, radiation therapy or surgery directed against solid tumors and for control of establishment of metastases. For example, the administration of therapeutically effective amounts of a combination of a PP2A activator and a BER inhibitor described herein may be conducted before, during or after chemotherapy, radiation therapy or surgery.

The phrase “combination therapy” embraces the administration of a combination of a PP2A activator and a BER inhibitor and/or additional further therapies as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents (i.e., a PP2A activator and a BER inhibitor), in a substantially simultaneous manner. Substantially simultaneous administration can be accomplished, for example, by administering to the subject a single capsule including a coformulation of a PP2A activator and a BER inhibitor having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a third and different therapeutic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

Thus, there is further provided a method of treating cancer comprising administering an effective amount of a PP2A activator and a BER inhibitor, or pharmaceutically acceptable salt forms thereof, to a subject, wherein a therapeutically effective amount of one or more additional cancer chemotherapeutic agents are administered to the patient. In some embodiments, administration of a PP2A activator and a BER inhibitor or pharmaceutically acceptable salt forms thereof, can restore sensitivity to one or more chemotherapeutic agents in a patient wherein the patient has developed a resistance to the one or more chemotherapeutic agents.

For the purposes of additional cancer chemotherapeutic agent therapy, there are large numbers of antineoplastic agents available in commercial use, in clinical evaluation and in pre-clinical development, which could be selected for treatment of cancers or other disorders characterized by rapid proliferation of cells by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents. Suitable agents which may be used in combination therapy will be recognized by those of skill in the art.

In some embodiments, the PP2A activator and the BER inhibitor can be administered to subject in combination with at least one anticancer agent that induces DNA damage in the cancer cells. Anticancer agents that induce DNA damage in the cancer cells include intercalating agents, such as bleomycin, adriamycin, quinacrine, echinomycin (a quinoxaline antibiotic), and anthrapyrazoles.

In some embodiments, radiotherapy can be used to induce DNA damage in the cancer cells. Radiotherapy may include ionizing radiation, particularly gamma radiation irradiated by commonly used linear accelerators or radionuclides. The radiotherapy by radionuclides may be achieved externally or internally. Radiotherapy may include brachytherapy, radionuclide therapy, external beam radiation therapy, thermal therapy (cryoablation hyperthermia), radiosurgery, charged-particle radiotherapy, neutron radiotherapy and photodynamic therapy, and the like.

Radiotherapy can be implemented by using a linear accelerator to irradiate the affected part with X-rays or an electron beam. While the X-ray conditions will differ depending on how far the tumor has advanced and its size and the like, a normal dose will be 1.5 to 3 Gy, preferably around 2 Gy, 2 to 5 times a week, and preferably 4 or 5 times a week, over a period of 1 to 5 weeks, for a total dose of 20 to 70 Gy, preferably 40 to 70 Gy, and more preferably 50 to 60 Gy. While the electron beam conditions will also differ depending on how far the tumor has advanced and its size and the like, a normal dose will be 2 to 5 Gy, preferably around 4 Gy, 1 to 5 times a week, and preferably 2 or 3 times a week, over a period of 1 to 5 weeks, for a total dose of 30 to 70 Gy, and preferably 40 to 60 Gy.

Treatment described herein can also be combined with treatments such as hormonal therapy, proton therapy, cryosurgery, and high intensity focused ultrasound (HIFU), depending on the clinical scenario and desired outcome.

Anticancer agents that induce DNA damage can also include DNA oxidizing agents, such as hydrogen peroxide.

Anticancer agents that induce DNA damage can further include alkylating agents such as Shionogi 254-S, aldo-phosphamide analogues, altretamine, anaxirone, Boehringer Mannheim BBR-2207, bestrabucil, budotitane, Wakunaga CA-102, carboplatin, carmustine (BiCNU), Chinoin-139, Chinoin-153, chlorambucil, cisplatin, cyclophosphamide, American Cyanamid CL-286558, Sanofi CY-233, cyplatate, dacarbazine, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, etoposide phosphate, fotemustine, Unimed G-6-M, Chinoin GYKI-17230, hepsul-fam, ifosfamide, iproplatin, lomustine, mafosfamide, mitolactol, mycophenolate, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, thiotepa, Yakult Honsha SN-22, spiromus-tine, Tanabe Seiyaku TA-077, tauromustine, temozolomide, teroxirone, tetraplatin and trimelamol.

Alkylating agents can function by adding methyl groups to DNA, cross-linking macromolecules essential for cell division, and linking guanine bases in DNA through their N⁷ atoms. Both inter- and intra-strand cross-links can be mediated by alkylating agents. Inter-strand cross-links prevent the separation of the DNA strands necessary for cell division, and by being more difficult to repair, constitute the more lethal lesion.

In certain embodiments, the anticancer agent is selected from radiosensitizers such as 5-iodo-2′-deoxyuridine (IUdR), fludarabine, 6-thioguanine, hypoxanthine, uracil, ecteinascidin-743, and camptothecin and analogs thereof.

The present invention is illustrated by the following examples. It is to be understood that the particular examples, materials, amounts, and procedures are to be interpreted broadly in accordance with the scope and spirit of the invention as set forth herein.

### Example

Small Molecule Activators of PP2A, (SMAPs), can potently inhibit polo-kinase 1 (Plk1), a major regulator of DNA damage response, and induce cell death in vitro and in vivo. Plk1 is both an attractive biomarker and a therapeutic target given that it is specifically overexpressed in cancer cells, including ovarian cancer where its expression correlates with histological grade and poor patient outcome. We show herein that SMAPs exert an anti-cancer effect through the dephosphorylation and degradation of Plk1. Activation of PP2A represents a highly novel approach to cancer treatment as it results in the coordinate downregulation of Plk1 and other key PP2A regulated oncogenic pathways, such as PI3K-AKT and MAPK.

In this example, we show that an orally bioavailable SMAP, SMAP-061, through activation of PP2A, downregulates key PP2A substrates such as Plk1, and confers anti-ovarian cancer activity. Furthermore, we show that combination SMAPs and PARP inhibitor treatment synergistically induce cancer cell death and decrease in vivo tumor burden, thus introducing a novel method to sensitize tumors to PARP inhibitors that rely on defective DNA repair machinery.

Results

SMAP-061 Induced Cell Death is Mediated Through the Negative Regulation of Plk1

DNA damage checkpoints are an important aspect of cell cycle progression, as they serve as a barrier for deleterious propagation of DNA damage to progeny cells by eliciting the DNA repair machinery. In cases where DNA damage exceeds repair capacity, cells are either withdrawn from the cell cycle or undergo cell death. On the other hand, when DNA damage is repaired, cells recover from cell cycle arrest in what is known as checkpoint recovery. One of the most important DNA damage checkpoints is G2/M, as it serves as the last DNA quality control to prevent genomic instability. Mitotic kinases such as Plk1 are essential for checkpoint recover, entry into mitosis and maintaining DNA integrity. Previous reports have reported overexpression of Plk1 in HGSOC tumors which also correlated with poor patient outcome.

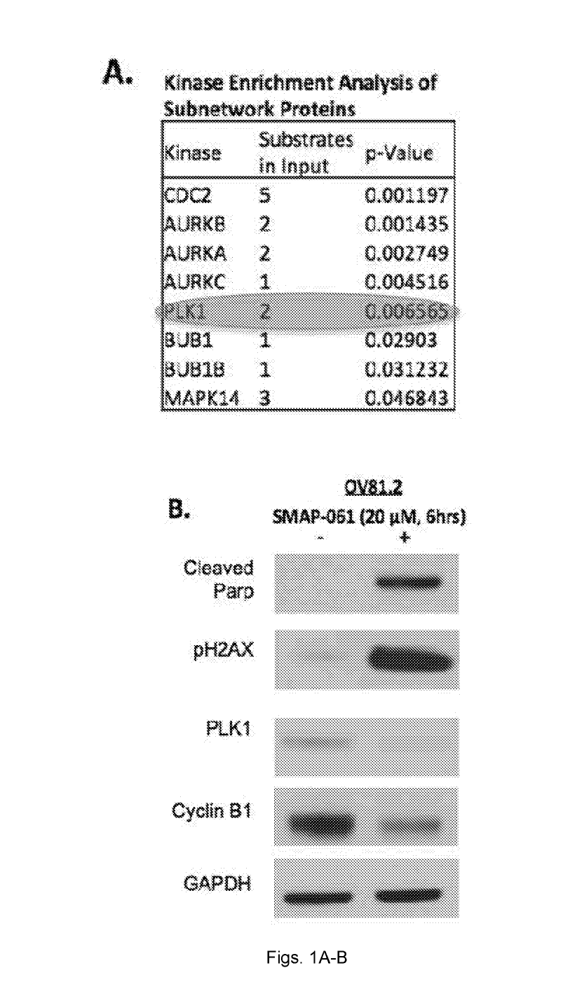

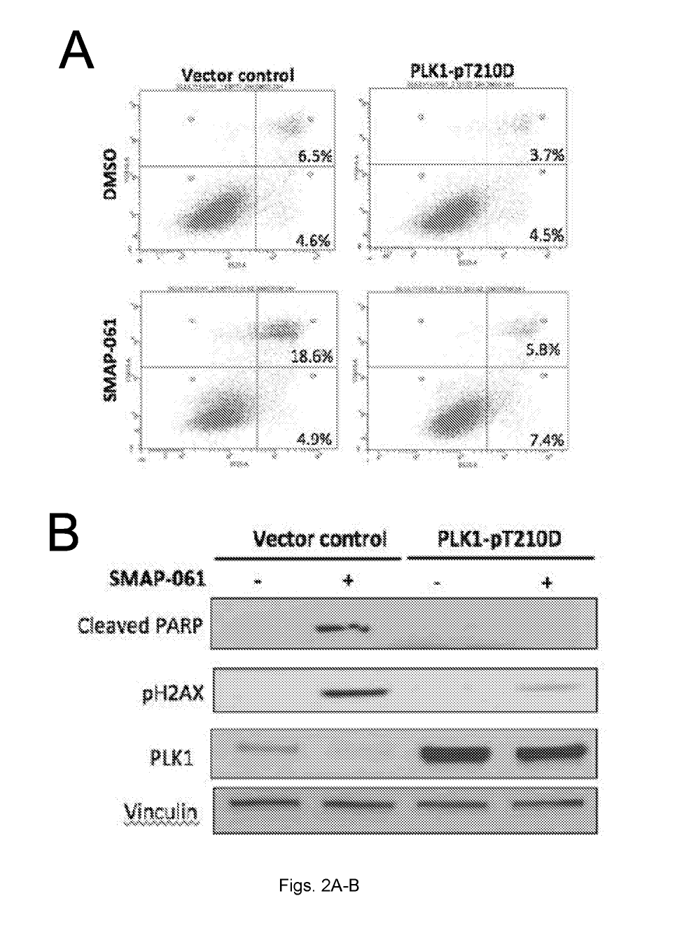

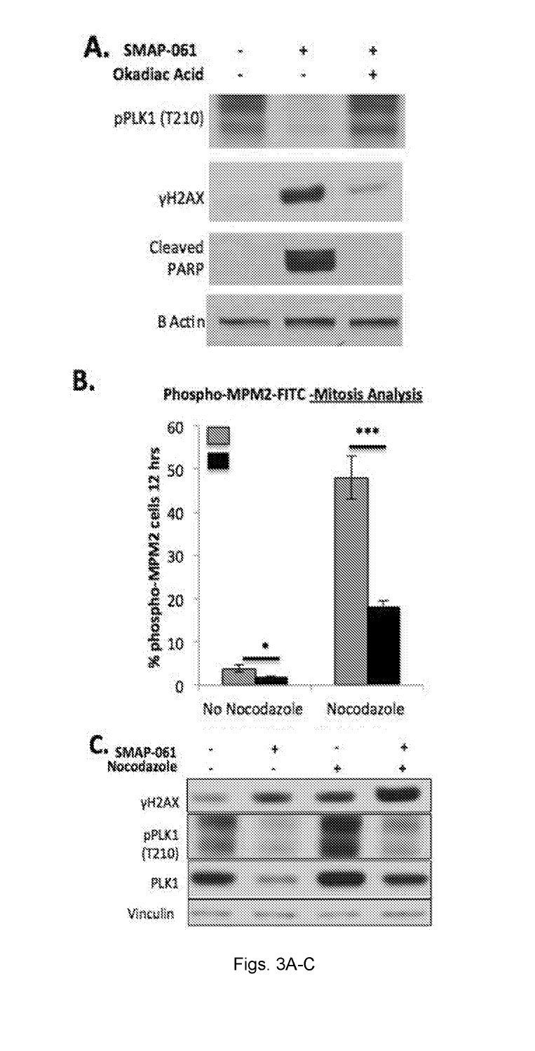

Additionally, given the high expression of Plk1 only in cancer cells and not normal cells, targeted approaches focusing on the inhibition of Plk1 are showing great promise in clinical trials for the treatment of various cancers but they have been plagued by hematologic toxicity and lack a specificity due to inhibition of Plk2 and Plk3 which have opposing functions to Plk122. Interestingly, these preclinical studies have also shown that Plk1 inhibitors may be active against tumors with mutations in TP53 which is a main alteration of >99% of HGSOC23. The major mode of Plk1 regulation involves its mitosis specific phosphorylation at threonine 210 which lies within the activation loop and plays an important role in stimulating Plk1 activity at the G2/M transition. Through a highthroughout phosphoproteomic anaylsis of cells treated with SMAPs we found that Plk1 at site T210 was significantly de-phosphorylated within 3 hours of SMAP treatment (FIG. 1A). Therefore, we sought to uncover whether SMAP-061 effects on cellular survival and apoptosis was mediated through Plk1 dephosphorylation. Interestingly, we found that primary ovarian cancer cells, treated with SMAP-061 (20 μM) for 6 hours resulted in the dephosphorylation and degradation of Plk1, as well as its downstream target cyclin B1 (FIGS. 1B, 3A & C). Furthermore, a dramatic induction of cleaved PARP and γH2AX was also observed (FIGS. 1B, 2A, 3A). However, in order to confirm that SMAP-061 effects on Plk1 was mediating the anti-tumor effects of the drug, we stably expressed a constitutively active form of Plk1 (T210D) which can no longer can be dephosphorylated by PP2A activation. Intriguingly, we discovered that the expression of T210D completely block SMAP-061 induction of apoptosis as measured by Annexin V staining (FIG. 2A). In addition, western blot analysis confirmed the abrogation of SMAP-061 induced apoptosis through the loss of cleaved PARP and γH2AX induction in the cells stably expressing the Plk1-T210D (FIG. 2B).

Furthermore, use of the PP2A pharmacological inhibitor, Okadaic acid (OA), eliminated SMAP-061 mediated degradation of Plk1 and concomitantly abolished the induction of apoptosis as detected by cleaved PARP and γH2AX, confirming that SMAP-061 activation of PP2A directly targets Plk1 for degradation and leads to apoptosis (FIG. 3A). Next, given that Plk1 is only expressed in dividing cells with peak expression during G2/M and PP2A tightly co-ordinates mitotic entry by negatively regulating Plk1, we performed nocodozale experiments which provokes increased expression of endogenous Plk1 through arresting cells in G2/M. We found that SMAP-061 treatment significantly blocked nocodazole induced M phase arrest as assessed by FACS analysis of Phopho-MPM2, a mitotic marker (FIG. 3B). It was also observed that treatment with SMAP-061 alone significantly decreased the percent of cells in M phase by >2-fold (FIG. 3B). Lastly, nocodozale mediated Plk1 induction was completely inhibited by SMAP-061 treatment and combining nocodazole and SMAP-061 further enhanced the induction of γH2AX (FIG. 3C).

Exploit SMAP-061 Effects on Plk1 and the DNA Damage to Sensitize Ovarian Tumors to PARP Inhibitors

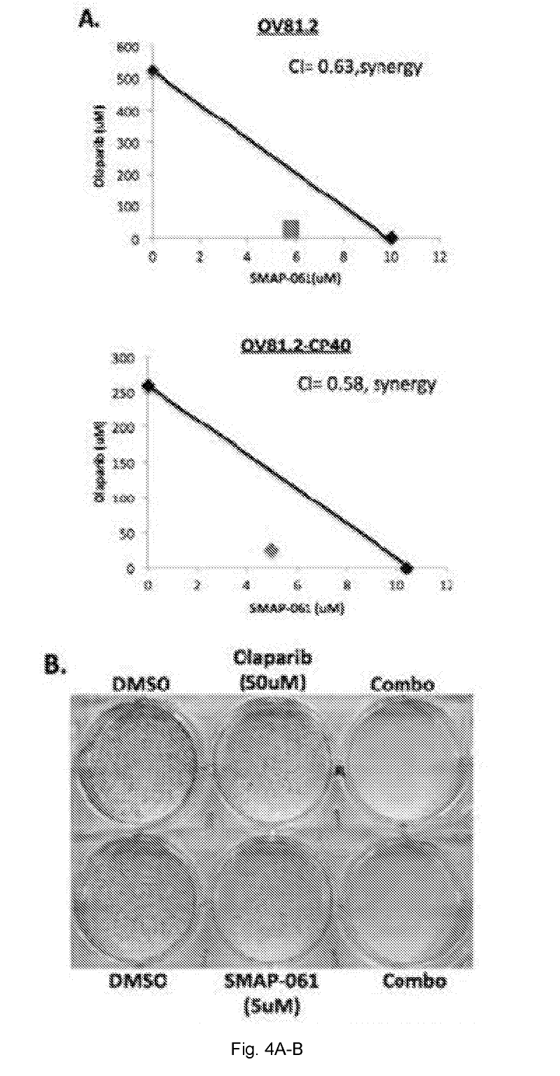

Alterations in the homologous repair pathway are thought to occur in 30%-50% of epithelial ovarian cancers. Cells deficient in homologous recombination rely on alternative pathways for DNA repair in order to survive, thereby providing a potential target for therapy. Olaparib, a poly(ADP-ribose) polymerase (PARP) inhibitor, capitalizes on this concept and is the first drug in its class approved for patients with HGSOC but unfortunately it is only approved for patients with germline BRCA1/2 mutation. Thus, there is a need to uncover methods to sensitize tumors to PARP inhibitors in the absence of BRCA1/2 mutations. Next in light of the recent FDA approval of PARP1 inhibitors for recurrent HGSOC treatment and given that the sensitization activity to PARP1 inhibitors are mediated through DNA damage response pathways such as Plk1, we examined whether SMAP-061 could sensitize cells to PARP inhibition. For these studies we used primary HGSOC cells line that are either sensitive or resistant to platinum therapy and are BRCA1/2 wildtype. Interestingly, we uncovered that in both cell lines the calculation of the combination index (CI) of cells treated with SMAP-061 and the PARP inhibitor, Olaparib, was significantly below 1.0, indicating a synergistic relationship between the two drugs (FIG. 4A).

Both cells lines were relatively unresponsive to Olaparib with IC₅₀ values between 250-500 μM, however in the presence of SMAP-061 the IC50 values for Olaparib dropped below 50 μM (FIG. 4A). Similar results were observed with another PARP inhibitor, veliparib (data not shown). Furthermore, colony formation assays also revealed that synergistic effects of the drugs on cellular survival (FIG. 4B). Lastly, western blot analysis revealed a marked increase in both cleaved PARP and γH2AX (which is currently the clinical biomarker for PARP inhibitors) in the combination treatment compared to each drug alone. In addition, there was a concomitant downregulation of Plk1 and cyclin B1 (FIG. 4C). Altogether, this data is highly relevant given the dire need to uncover methods to overcome PARP inhibitor resistance and expand the use of this novel drug to tumors that are BRCA1/2 wildtype.

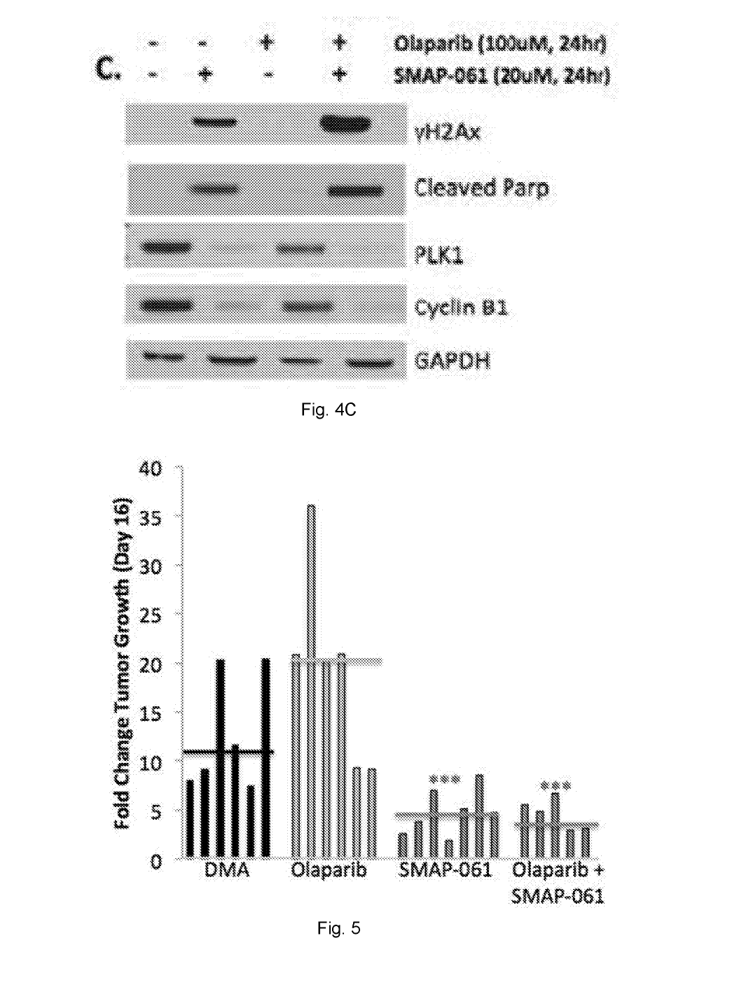

Next we performed combination drug studies in HGSOC PDX models. The tumor studies were carried out as follows: when tumorgrafts reach approximately 150 mm³, animals were randomized and dosing was initiated based upon preliminary studies in PDX OV81 (BRCA1/2 wildtype): DMA control, SMAP-061 (15 mg/kg, twice/day everyday by oral gavage), Olaparib (100 mg/kg every other day) or SMAP-061 (15 mg/kg, twice/day everyday by oral gavage)+Olaparib (100 mg/kg every other day). We found that SMAP-061 dramatically sensitized tumors that do not respond to Olaparib (FIG. 5).

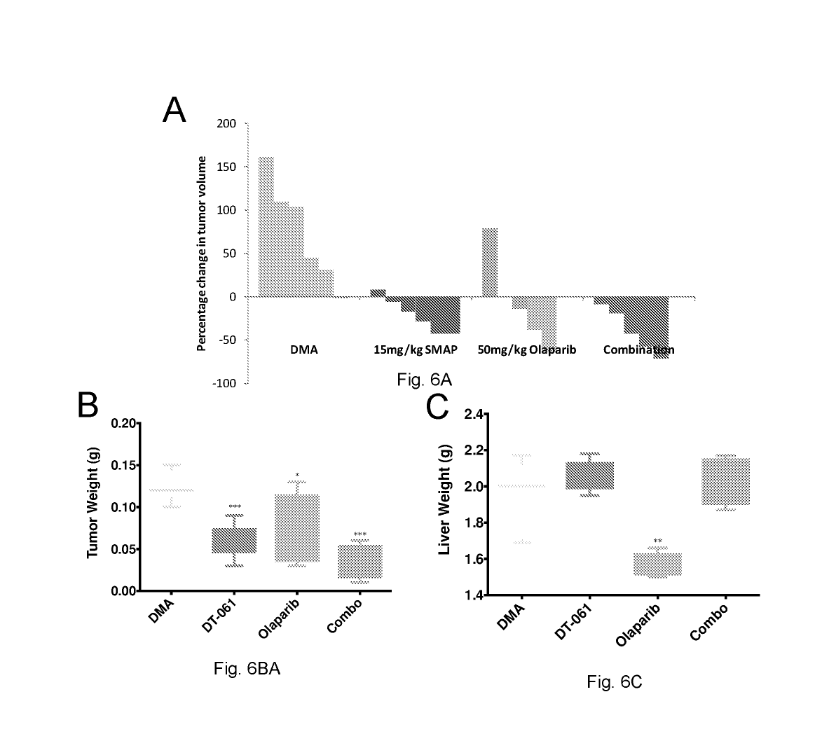

We performed combination drug studies in PDX:O17 models (germline BRCA1, high grade serous ovarian cancer patients). The tumor studies were carried out as follows: when tumorgrafts reach approximately 150 mm³, animals were randomized and dosing was initiated based upon preliminary studies in PDX OV81 (BRCA1/2 wildtype): DMA control, SMAP-061 (15 mg/kg, twice/day everyday by oral gavage), Olaparib (50 mg/kg every other day) or SMAP-061 (15 mg/kg, twice/day everyday by oral gavage)+Olaparib (50 mg/kg every other day). The data confirms the synergistic effects PP2a activator and PAPR inhibitor (olaparib). FIGS. 6(A-C) show the change in tumor volume at the end of the study, as well as the tumor weights and liver weights for toxicity. Interestingly, the PARPi arm does show a decrease in liver weight however when the drugs are combined with PP2A activator this is eliminated (FIG. 6C).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method for treating cancer in a subject in need thereof comprising:
administering to the subject therapeutically effective amounts of a PP2A activator and BER inhibitor.

2. The method of claim 1, wherein the subject is administered a pharmaceutical composition including a coformulation of the PP2A activator and the BER inhibitor.

3. The method of claim 1, wherein the cancer is characterized by cancer cells in which PP2A has reduced activity.

4. The method of claim 1, wherein the cancer is characterized by cancer cells in which Plk1 is overexpressed.

5. The method of claim 1, wherein said cancer is high grade serious ovarian cancer.

6. The method of claim 1, wherein the subject has BRCA genotype not associated with an increased risk of hereditary breast-ovarian cancer syndrome

7. The method of claim 1, wherein the subject has a BRCA genotype associated with an increased risk of hereditary breast-ovarian cancer syndrome.

8. The method of claim 1, wherein the PP2A activator is a small molecule.

9. The method of claim 8, wherein the PP2A activator is a trycyclic neuroleptic compound or derivative thereof.

10. The method of claim 1, wherein the PP2A activator is a tricyclic neuroleptic compound devoid of GPCR or monoamine transporter pharmacology.

11. The method of claim 1, wherein the BER inhibitor is a PARP inhibitor.

12. The method of claim 11, wherein the PARP inhibitor is a PARP1 inhibitor selected from group consisting of nicotinamide; NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1(2H)-pyridinyl)propyl]-4(3H)-quinazo-linone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5[4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281; ABT-888 (Veliparib); BSI-201 (Iniparib); Rucaparib (AG-014699); INO-1001; A-966492; PJ-34 is an EGFR inhibitor selected from erlotinib, gefitinib, lapatinib, and icotinib.

13. A method for treating cancer in a subject in need thereof comprising:
administering to the subject therapeutically effective amounts of a PP2A activator and a PARP inhibitor.

14. The method of claim 13, wherein the subject is administered a pharmaceutical composition including a coformulation of the PP2A activator and the BER inhibitor.

15. The method of claim 13, wherein the cancer is characterized by cancer cells in which PP2A has reduced activity.

16. The method of claim 13, wherein the cancer is characterized by cancer cells in which Plk1 is overexpressed.

17. The method of claim 13, wherein said cancer is high grade serious ovarian cancer.

18. The method of claim 13, wherein the subject has BRCA genotype not associated with an increased risk of hereditary breast-ovarian cancer syndrome

19. The method of claim 13, wherein the subject has a BRCA genotype associated with an increased risk of hereditary breast-ovarian cancer syndrome.

20. The method of claim 13, wherein the PP2A activator is a small molecule.

21. The method of claim 13, wherein the PP2A activator is a trycyclic neuroleptic compound or derivative thereof.

22. The method of claim 13, wherein the PP2A activator is a tricyclic neuroleptic compound devoid of GPCR or monoamine transporter pharmacology.

23. The method of claim 13, wherein the PARP inhibitor is a PARP1 inhibitor selected from group consisting of nicotinamide; NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1(2H)-pyridinyl)propyl]-4(3H)-quinazo-linone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5[4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281; ABT-888 (Veliparib); BSI-201 (Iniparib); Rucaparib (AG-014699); INO-1001; A-966492; PJ-34 is an EGFR inhibitor selected from erlotinib, gefitinib, lapatinib, and icotinib.
